(19) United States
(12) Patent Application Publication
(10) Pub. No.: US 2011/0311533 A1
(43) Pub. Date: Dec. 22, 2011

(54) REGULATION OF PROTEIN LEVEL

(76) Inventor: Marcela Karpuj, San Francisco, CA (US)

(21) Appl. No.: 13/103,481

(22) Filed: May 9, 2011

Related U.S. Application Data
(60) Provisional application No. 61/333,018, filed on May 10, 2010.

(51) Int. Cl. A61K 39/395; A61P 43/00; A61K 38/17; A61K 31/7088; C12N 5/071; C07K 19/00

(52) U.S. Cl. 424/134.1; 435/375; 530/387.3; 514/21.2; 514/44 R

(57) ABSTRACT

The invention relates to regulating the level of a membrane-anchored target molecule using PrP or a fragment thereof and a polyanion. Direct or indirect methods for targeting protein downregulation, mediated through PrP or a fragment thereof and a polyanionare are described.

This represents a method for treating/preventing a disease, disorder or condition comprising administration to a subject in need of a therapeutically amount of a chimeric protein comprising PrP or a fragment thereof and a protein of interest, the DNA encoding the chimeric protein, the vector encoding the DNA or host cell harboring the DNA encoding the chimeric protein and a subsequent polyanion administration. It provides a pharmaceutical composition comprising a carrier and a chimeric protein comprising PrP or a fragment thereof fused to a protein of interest, the DNA encoding the chimeric protein, the vector encoding the DNA or host cell harboring the DNA encoding the chimeric protein.

This Non-Provision Application claims benefit of the priority of the filing date 10-MAY 2010 of the Provisional 61333018 by Marcela Karpuj.

### FIELD OF THE INVENTION

The invention relates to PrP and fragments thereof and to their use in protein level regulation.

### BACKGROUND OF THE INVENTION

Prion disease is a fatal neurodegenerative disorder caused by the conversion of the cellular prion protein (PrPC) into its infectious conformation, PrPSc. Accumulation of PrPSc in neurons is associated with neurodegeneration and disease progression but the exact mechanism leading to neurodegeneration and death is still unknown. Therefore, efforts are directed towards the elucidation of the biological function of PrPC, hoping that this might shed a light on the course of prion disease.

PrP is expressed in other cell types besides neuronal cells but the infectious protein, PrPSc, was found mainly in the neuropil and the reason for that specific localization is still unknown (S. J. DeArmond et al. 1987). PrP is found in several cell compartments such as plasma membrane, cytosol and in the nucleus (Gabus et al. 2001). It is expressed on T cells, B cells, monocytes, NK cells, platelets and even on beta cells (Atouf et al. 1994)(Vostal et al. 2001). Moreover, it is a very conserved protein throughout evolution, implying a possible important biological role (Rivera-Milla et al. 2005).

Interestingly, mice lacking PrP display a normal development and behavior (Bileler et al. 1992). Nevertheless, PrP was demonstrated to be necessary for normal synaptic function and seems to have a role in the regulation of embryonic cell adhesion in Zebrafish (Collinge et al. 1994)(Málaga-Trillo et al. 2009). Recently it was shown that ablation of cellular protein expression affects Mitochondrial numbers and morphology (Miele et al. 2002).

Other Physiological functions of PrP were postulated from its interacting partners. PrP binds N-CAM, laminine receptor, glutathione S-transferase, Hsp60 and GroEL, providing the assumption that PrP might be involves in some sort of cell signaling (Schmitt-Ulms et al. 2001)(Roman Rieger et al. 1997)(F Edenhofer et al. 1996). PrP, binds polyanions such as nucleic acids and sulfated Glycans, and other small molecules such as copper and chloroquine (Brimacombe et al. 1999)(Stockel et al. 1998)(Vogtherr et al. 2003). Despite of intensive efforts trying to elucidate the normal function of PrP, there is no conclusive evidence so far.

Interestingly, some of these molecules are known to eliminate PrPSc infectivity in cells and in animals infected with prions (Karpuj et al. 2007)(Barret et al. 2003). Phosphorothioates DNA (PS-DNA) (stabilized DNA analogs showing reduced enzymatic degradation) down-regulates levels of both PrPSc and PrPC, in cell cultures (Karpuj et al. 2007). It has been demonstrated that PS-DNA has no effect on PrP transcription or translation (Karpuj et al. 2007). Moreover, PrP degradation is probably occurring at the lysosome, since lysosomal inhibitors, but not proteosomal inhibitors, prevented PrP degradation in the presence of PS-DNA. It is important to note that single strand PS-DNA and double strand PS-DNA were equally able to degrade PrPSc, but not PS-RNA. Moreover, any PS-DNA sequence composed of 18-mers, 22-mers and 44-mers abolished PrPSc (Karpuj et al. 2007). Kociscos et al demonstrated, independently, that PS-DNA binds PrPC, and leads to its increased internalization. All groups discovered, independently, that PS-DNA leads to PrPSc reduction in cell cultures, and prolongs survival of animals infected with PrPSc (Karpuj et al. 2007)(Kocisko et al. 2006)(Prusiner S B. 2004).

### SUMMARY OF THE INVENTION

The invention relates to regulating the level of a membrane-anchored target molecule (e.g. a protein of interest) using PrP or a fragment thereof and a polyanion.

In one aspect, the invention relates to a method for regulating the level of a membrane-anchored protein of interest in a cell comprising the steps of

A—i) Introducing in the cell a chimeric protein comprising the protein of interest and prion protein (PrP) or a fragment thereof; or

ii) Exposing the cell to a chimeric protein comprising a protein of interest-binding molecule and PrP or a fragment thereof, and

B—Exposing the cell to a polyanion.

In one embodiment of the invention, the PrP fragment is the N-terminus of PrP or a fragment thereof.

In another embodiment of the invention, the protein of interest-binding molecule is a specific antibody raised against the protein of interest.

In a further embodiment of the invention, the protein of interest is a natural membrane protein.

In a further embodiment of the invention, the polyanion is PS-DNA.

In another aspect, the invention relates to a chimeric protein comprising PrP or a fragment thereof fused to a molecule capable of binding a membrane-anchored protein of interest.

In one embodiment of the invention, the binding protein is an antibody specific to the protein of interest.

In another aspect, the invention relates to a method for treating or preventing a disease, disorder or condition caused or augmented by unregulated levels of a membrane-anchored protein of interest comprising administration to a subject in need of

A—i) A therapeutically effective amount of a chimeric protein comprising prion protein (PrP) or a fragment thereof and the protein of interest, the DNA encoding the chimeric protein, or the vector encoding the DNA, or ii) A therapeutically effective amount of a chimeric protein comprising a protein of interest binding molecule and PrP or a fragment thereof, the DNA encoding the chimeric protein, the vector encoding the DNA; and

B—Subsequent administration of a therapeutically effective amount of polyanion.

In one embodiment of the invention, the binding molecule is a specific—antibody raised against the protein of interest.

The method allows regulating the expression level of the protein of interest in vivo or ex vivo. The method can be used with a eukaryotic cell such as a stem cell.

In another aspect, the invention relates to a method for treating or preventing a disease, disorder or condition comprising administration to a subject in need of a therapeutically effective amount of a chimeric protein comprising PrP or a fragment thereof and a protein of interest, the DNA encoding the chimeric protein, the vector encoding the DNA or host cell harbouring the DNA encoding the chimeric protein and a subsequent administration of a polyanion.

Also, the invention provides a pharmaceutical composition comprising a pharmaceutically acceptable carrier and a chimeric protein comprising PrP or a fragment thereof fused to a protein of interest, the DNA encoding the chimeric protein, the vector encoding the DNA or host cell harbouring the DNA encoding the chimeric protein.

The invention provides a chimeric protein comprising PrP or a fragment thereof fused to a molecule capable of binding a protein of interest, the DNA encoding the chimeric protein, the vector encoding the DNA or host cell harboring the DNA encoding the chimeric protein.

The invention also provides a chimeric protein comprising PrP or a fragment thereof fused to a fluorescent protein at its C-terminus and to a different fluorescent protein at its N-terminus.

In one embodiment, the fluorescent proteins are CFP and YFP.

The invention additionally provides a kit comprising a polyanion and chimeric protein comprising PrP or a fragment thereof and one or more proteins of interest, the DNA encoding the chimeric protein, the vector encoding the DNA or host cell harbouring the DNA encoding the chimeric protein.

In one embodiment, the protein of interest is a fluorescent protein fused to the PrP or fragment thereof at its C-terminus.

In another embodiment, the PrP or fragment thereof in the chimeric protein is fused to one fluorescent protein at its C-terminus and to another fluorescent protein at its N-terminus.

### BRIEF DESCRIPTION OF THE FIGURES

FIG. 1 Shows the effect of PS-DNA on Native PrP as well as transfected PrP in various cell types from different origin. Different cell lines were exposed to 10 μM PS-DNA for 24 h. Native or transfected PrP are down-regulated in various cell lines exposed to PS-DNA. (a) Western blot analysis of N2a (mouse neuroblastoma), TSM (mouse neurocortical neuroblasts), CHO-3F4PrP (Chinese hamster ovary cells stably transfected with mouse PrP containing the 3F4 PrP epitope), ScGT-3F4PrP (scrapie-infected mouse hypothalamic neuronal cell line stably transfected with 3F4PrP) and HEK 293 cells (human embryonic kidney). PrP was detected using the D13 (lanes 1-4,7, and 8) or the 3F4 (lanes 5,6,9, and 10) anti-PrP antibody (b) Densitometic quantification of Western blot analyses (n=3 independent experiment, p=0.001).

FIG. 2. Shows that PrP glycosylation has no effect on PS-DNA induced down-regulation. (a) Western blot analysis of N2a cells stably transfected with the three indicated PrP plasmid constructs differing in their glycosilation level. Transfected PrP was detected using the 3F4 monoclonal antibody, and the endogenous mouse PrP was detected using the D13 antibody. (b) Densitometric quantification of Western blot analyses. (c) Schematic representation of point mutations examined (n=3 independent studies, p=0.01).

FIG. 3 shows that PS-DNA treatment results in reduction of existing as well as newly synthesized PrP. (a) Western blot analysis of N2a cells treated with or without 1.5 mg/ml tunicamycin for the indicated times and PS-DNA. PrP was detected using the D13 antibody. (b) Densitometric quantification of Western blot analyses. Black bars represent the percentage of non-glycosilated PrP, containing mostly the newly synthesized PrP (3a, lower band in lane 1, 3, and 5; compared to lanes 2, 4, and 6 respectively). Gray bars represent the percentage of PrP in cells that were not treated with tunicamycin but were exposed to PS-DNA (3a, lanes 7, 9, and 11 as a percentage of lanes 8, 10, and 12 respectively). White bars represent the percentage of existing (fully glycosylated) PrP after exposure to PS-DNA (n=3 independent studies, p=0.04).

FIG. 4 Shows that PrP N-terminus is essential for PrP down-regulation in the presence of PS-DNA. (a and b) Western blot analysis of PS-DNA treated N2a cells transiently transfected with various deletion mutated forms of PrP. (c) Schematic representation of PrP. SS-signal sequence, PO-pre octarepeat domain, OR-octarepeat domain, GPI-glycosylphosphatidylinositol anchor. Deletions are indicated as open boxes containing the aa position of their boundaries. (d) Densitometric quantification of Western blot analyses (n=3 independent studies, p=0.009 for mutant 1 and 12, p=0.0005 for MHM2, mutant 3, mutant 2, and p=0.0008 for MoXenPrP).

FIG. 5 Shows that PrP reduction by PS-DNA is transient. (a) Western blot analysis of N2a cells exposed to PS-DNA followed by recovery periods. Untreated N2a cells were harvested after 4, 8 and 28 hours (lanes 1, 5, and 8 respectively). (b) Densitometric quantification of Western blot analyses. (n=3 independent studies, p=0.04)

FIG. 6 shows that Nuclear, cytosolic and mitochondrial PrP are not down-regulated in cells exposed to PS-DNA. (a) Western blot analysis of N2a cells transiently transfected with plasmid-constructs directing PrP into different cellular compartment. (b) Densitometric quantification of Western blot analyses (n=3 independent studies).

FIG. 7 shows Different membrane anchoring modalities do not affect PS-DNA induced PrP down-regulation. (a) Western blot analysis of N2a cells transiently transfected with the indicated plasmid constructs. PrP-CD4, fusion of PrP aa 1-230 and the transmembrane domain of mouse CD4 (aa369-431); PrP-Thy1, fusion of PrP (aa1-230) and the GPI anchor of Thy1 (aa 127-163); NT-Thy-1, fusion of aa 1-22 of PrP, the flag epitop, PrP N term (aa 23-88), and Thy1. Thy1 was detected using anti HA antibodies, NT-Thy1 was detected using anti Flag antibodies. All other PrP forms were detected using the 3F4 monoclonal antibody. (b) Densitometric quantification of Western blot analyses (n=3 independent studies, p=0.0001, p=0.005, p=0.0004 for 3F4PrP, PrP-Thy1, and for NT-Thy1 respectively).

FIG. 8. Membrane-anchored PrP is down-regulateded in response to PS-DNA treatment. Western blot analysis of media or cellular fractions of N2a cultures exposed to PS-DNA for 72 hours (Lanes 1, 2, 5 and 6) and treated with 200 mM PIPLC for 6 hours. The media of the cultures were TCA precipitated (Lanes 1-4). PrP was detected using the D13 antibody (n=3 independent studies, p=0.001).

FIG. 9 shows that Attachment of PrP at the N-terminus of the Dpl protein results in susceptibility to PS-DNA. (a) Western blot analysis of N2a cells transiently transfected with different fusion products of PrP and Dpl. (b) Densitometric quantification of Western blot analyses (n=3 independent studies, p=0.04). (c) Schematic representation of the fusion products examined. The aa position of the boundaries of PrP (gray boxes) and Dpl (dark boxes) are indicated in each case. Significance of the various marked domains of PrP is indicated in legend to FIG. 2.

FIG. 10 Attachment of PrP at the C-terminus of the cytosolic proteins YFP and CFP promotes PS-DNA susceptibility. (a) Western blot analysis of CHO cells transiently transfected with CFP-PrP or YFP-PrP fusion products and treated with PS-DNA. (n=2 independent studies) (b) Densitometric quantification of Western blot analyses in panel a. (c) Western blot analysis of N2a cells transiently transfected with CFP-PrP or YFP-PrP fusion products and treated with PS-DNA. (d) Position of YFP or CFP insertion in PrP.

FIG. 11 shows the numbering of the different epitopes on the deletion mutantchimeras used. MHM2, PrP mouse protein with the hamster epitope 109-112; Dpl, Dopel protein; Thy1, NT-Thy1 ; PrP aa 1-22 plus the HA tag plus PrP aa 23-88 tagged to full length Thy1 on its N terminus; SS, Signal sequence of PrP; PO, Pre-octarepeat area; OR. Octarepeat are in PrP; CHO, glycosilation site; Star on CHO, Mutation of the glycosilation site; GPI-Glycosylphosphatidylinositol anchor site; HA, HA tag; FLAG, FLAG tag; D13, recognition site of D13 antibody; 3F4, recognition site by the antibody 3F4. Areas in red are the ones that are deleted. For more details see materials and methods (iii) Constructs, transformation, and primers.

FIG. 12 Shows different combinations in which the method of the invention could be used to regulate target molecules or membrane anchored proteins of interest. A: In one embodiment the target protein is a cell surface protein recognized by an antibody fused to PrP or PrP fragment through its Fc region and when exposed to polyanion the levels of the target protein is downregulated. In another embodiment the target molecule or protein of interest on the cell surface is a glycoprotein. B: In one embodiment an antibody specific to another membrane protein is fused to PrP or PrP fragment and to the target protein of interest. This method will increase first the basal levels of the target protein but when exposed to the polyanion this level will be reduced. C: In one embodiment instead of using an antibody to connect between PrP and the target protein, any other molecule that binds the cell surface could be used. This molecule could bind directly to the target molecule as described in C or indirectly as described in B. Some examples of binding molecules are receptor ligands or small molecules that bind cell surface molecules. 1) The target protein. 2) Antibody recognizing the target protein. 3) Antibody recognizing the target protein and fused to PrP or PrP fragment. 4) Antibody recognizing other cell membrane anchored protein fused to PrP or PrP fragment and to the target protein. 5) Small molecule or binding protein fused to PrP or PrP fragment.

### DETAILED DESCRIPTION OF THE INVENTION

The invention relates to regulating the level of a membrane-anchored target molecule (e.g. a protein of interest) using PrP or a fragment thereof and a polyanion.

In one aspect, the invention relates to a chimeric protein comprising PrP or a fragment thereof fused to one or more proteins and to its use.

The invention relates to a protein comprising PrP or a fragment thereof fused to another protein and to its use. The fusion can be done though chemical modification or using genetic engineering expression methods.

The PrP according to this invention can be the full length of PrP or comprise fragments of PrP. PrP fragments can be amino acids (aa) 23-230 or fractions of a shorter version of N terminus PrP such as aa 23-51, aa48-92 or aa 68-92. PrP or fragments of PrP can include all vertebrate PrP such at Zebra Fish, Xenopus, Turtle, Chicken, Hamster, Mouse, human, Pig and Dog PrP or fragments thereof PrP includes homologous proteins. Two or more structures are said to be homologous if they are alike because of shared ancestry. Homology among proteins and DNA is often concluded on the basis of sequence similarity, especially in bioinformatics. For example, in general, if two genes have an almost identical DNA sequence, it is likely that they are homologous. Many algorithms exist to cluster protein sequences into sequence families, which are sets of mutually homologous sequences. Homology of sequences can be of two types: orthologous or paralogous. Two similar genes in two different species that originated from a common ancestor are orthologous. Homologous sequences are orthologous if they were separated by a speciation event: if a gene exists in a species, and that species diverges into two species, then the divergent copies of this gene in the resulting species are orthologous. A second definition of orthologous describes any two genes in two different species with very similar functions. Homologous sequences are paralogous if they were separated by a gene duplication event: if a gene in an organism is duplicated to occupy two different positions in the same genome, then the two copies are paralogous. The genes encoding myoglobin and hemoglobin are considered to be ancient paralogs.

The target molecule or protein of interest according to the invention can be any membrane protein that is on the cell surface such as in the case of a receptor and ion channels. It can be prokaryotic or eukaryotic proteins such as, but not limited to, transport proteins, ion channels, ligand receptors, G-protein coupled receptors, motor and motor associated proteins, growth factor receptors, and cytoskeletal structural components. Also, proteins involved in the function of sensory and neural functions are considered according to the invention. In addition, membrane proteins from numerous viral and microbial organisms can be used. Also, eukaryotic host cell receptor ligands. Prokaryotic proteins would include for example microbial specific factors such as muscin, and other potential proteins serving as identifiable markers for middle ear infections. The protein used can be involved in disease associated with this transport such as in cystic fibrosis and peroxisomal biogenesis disorders; carbohydrate metabolism and its hormonal control; diabetes mellitus; hormone receptors and signal transduction; endocrine disorders; normal and abnormal processes of lipid, protein, amino acid, urea, pyrimidine, metal ion and steroid metabolism; and genetic metabolic disorders. Proteins/enzymes involved in the response of cells to environmental toxicants can be used. These proteins/enzymes may include the components of the stress signaling pathway, ion channels involved in transport of xenobiotics (e.g., membrane transporters as PgP and MDR, MRP2, transporters and enzymes responsible for the uptake metabolism and clearance of environmental toxicants, targets of toxicant action including the Ah receptor nonclassical receptors for endocrine disrupting agents, and membrane bound heat shock proteins). These proteins may include neurotransmitter and growth factor receptors, transporters, ion pumps, voltage- and ligand-gated ion channels, trafficking proteins, mitochondrial proteins, structural proteins and other proteins involved in the normal function and pathology of cells (neurons and glia) in the central and peripheral nervous system. Of interest are proteins involved in synaptic transmission and in the regulation, metabolism, and homeostasis and signaling in the brain during functions such as learning and memory or cognition. These can be also proteins that are not expressed normally on the cell surface but can be targeted to the cell surface by tagging them with a sequence, that will lead to their membrane anchoring such as GPI anchoring or a transmembrane domain. The protein of interest can be targeted to the cell membrane by using an antibody specific to a membrane protein. Considered are these proteins or partial sequence of these proteins including structural or regulatory proteins. The protein of interest can be a protein whose level correlates with a disease. For example, decreasing geranylgernylated RhoA and farnesylated Ras at the plasma membrane leading to autoimmunity modulation (Dunn et al., 2006).

In one embodiment of the invention, the target molecule protein is a cell surface protein recognized by an antibody fused to PrP or PrP fragment through its Fc region and when exposed to polyanion the levels of the target protein is downregulated (FIG. 12). In another embodiment, the target molecule or protein of interest on the cell surface is a glycoprotein. In another embodiment, an antibody specific to another membrane protein is fused to the PrP, or PrP fragment and to the target protein of interest. This method will increase first the basal levels of the target protein but when exposed to the polyanion this level will be reduced (FIG. 12B). In another embodiment, instead of using an antibody to connect between PrP and the target protein, any other molecule that binds the cell surface could be used. This molecule could bind directly to the target molecule as shown in FIG. 12C or indirectly as shown in FIG. 12B. Some examples of binding molecules include, but are not limited to, receptor ligands or small molecules that bind cell surface molecules.

The term “regulating expression level of the protein” relates for example to decreasing the level of a protein. This decrease can be transient or continued. Regulating the expression level of the protein according to this invention can be carried out by keeping the level of the protein constant, down-regulated or up-regulated.

In one embodiment of the invention, a PrP tagged antibody or small molecule capable of binding the T cell Receptor (TCR) present on a cell is used together with the polyanion to down regulate the TCR levels in the cell as long as the polyanion is present.

In another embodiment of the invention, the method is used to regulate proliferation or differentiation of stem cells by introducing a regulatory protein on the cell surface of the stem cells ex vivo by transfection of that protein. The regulatory protein may be a membrane protein or a non membrane protein fused to an anchoring molecule. In these embodiments the regulatory protein is fused to PrP or fragments of PrP. When desired, the protein levels are downregulated by introduction of the polyanion. The downregulation of the regulatory protein persist as long as the polyanion is present in the system. Example of such regulatory proteins could be Scrib, LRP5/6, Frizzled, or GFR (Steelman, Chappell, McCubrey, & Abrams, 2011). In one embodiment, the target protein can be an ion channel, receptor, ligand, hormone, a modified protein, glycolipid, lipid, sugar etc. (FIG. 14).

The host cell according to this invention can be a prokaryotic cell or an eukaryotic cell and a cell hosting a new protein, or DNA. The cells can be cell lines or primary cells or tissue cells. They can be single isolated cells or different types of cells. The cell can be a cell that harbors foreign molecules, viruses, or microorganisms, for example, a cell being host to a virus. An organism that harbors a parasite (that is, a virus, a bacterium, a protozoa, or a fungus), or a mutual or commensal symbiont.

The polyanion according to the invention can be any a molecule or chemical complex having negative charges at several sites such as DNA, RNA. It can be any DNA with any sequence, and any modifications on the backbone for example a phosphorodie ster or phosphorothioate.

The N-terminus of PrP according to this invention can be any region on PrP that comprises the portion of aa 1-92, bigger than that or smaller. This part can contain different tags in it or at the edges, and different modifications.

The term fragment refers to any subset of the molecule, that is, a shorter peptide that retains the desired activity of PrP such as the activity of protein level regulation. Fragments may readily be prepared by removing amino acids from either end of PrP, and testing the resultant fragment for its activity. Proteases for removing one amino acid at a time from either the N-terminal or the C-terminal of a polypeptide are known, and so determining fragments, which retain the desired activity, involves only routine experimentation. Fragments can be prepared by genetic engineering as shown in the examples.

In one embodiment of the invention, the PrP fragment is one corresponding to a N-terminal fragment of a PrP. PrP fragments include muteins thereof. The present invention further covers any fragment or precursors of the polypeptide chain of the protein molecule alone or together with associated molecules or residues linked thereto, e.g., sugar or phosphate residues, or aggregates of the protein molecule or the sugar residues by themselves, provided said fraction has substantially similar activity to PrP.

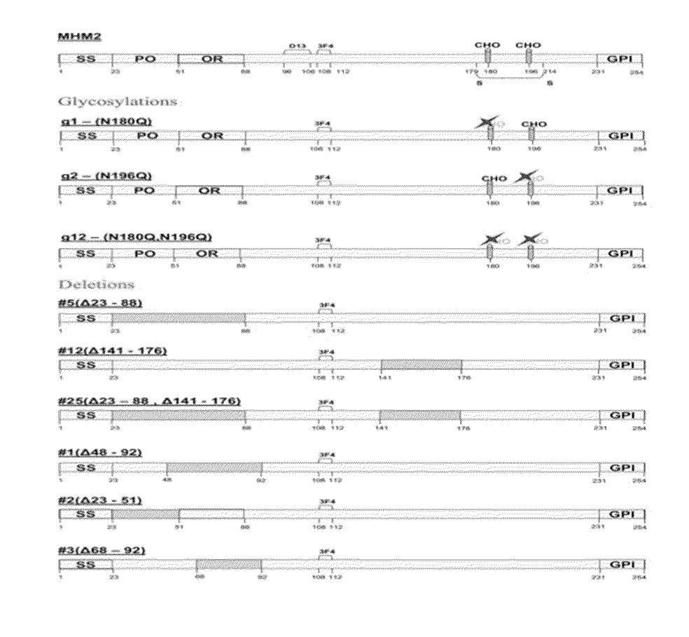

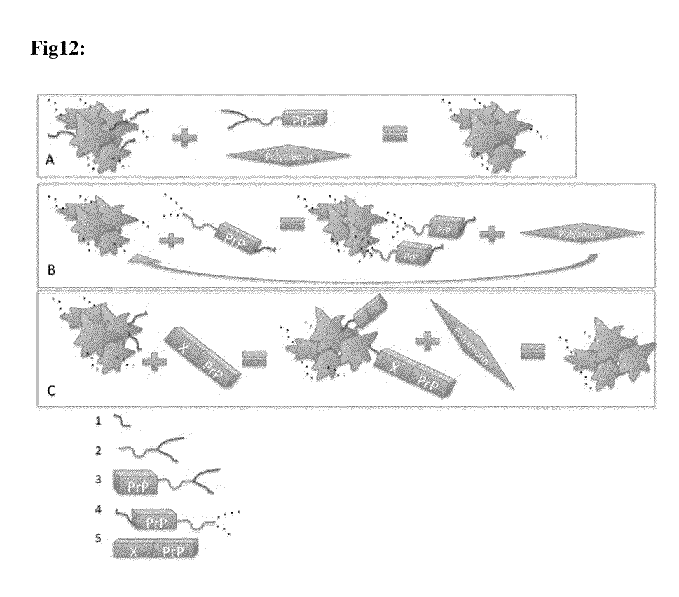

The term “fused protein” refers to a protein comprising a PrP, or a fragment thereof, fused with another protein, which, e.g. a membrane protein. This fusion could be done chemically using a coupling agent. This cross-linking is in the presence of a bifunctional reagent in a condition that keeps the proteins in their native form. For more detailed example see the book Antibodies a Laboratory Manual by Ed Harlow and David Lane p 129. Another option is to create a vector encoding the fusion protein as described in FIG. 10 and FIG. 11 and introduced into the system. The system could be for example a cell, organ, or an organism.

“Isoforms” of PrP are proteins or fragments capable of having PrP activity or fragment thereof, which may be produced by alternative splicing.

The tag according to this invention can be any sequence that can be recognized by an antibody or any other molecule in a specific manner. It can be any molecule that will allow the identification or isolation of the tagged protein. For example it can be a fluorescent protein or a synthetic peptide such as Strep-tag. This is a synthetic peptide consisting of eight amino acids (Trp-Ser-His-Pro-Gln-Phe-Glu-Lys). This peptide sequence exhibits intrinsic affinity towards Strep-Tactin, a specifically engineered streptavidin and can be N- or C-terminally fused to recombinant proteins. By exploiting the highly specific interaction, Strep-tagged proteins can be isolated in one step from crude cell lysates.

The term “in vivo” according to the invention can be any type of animal from different species. It includes experimentation using any whole, living organism as opposed to a partial or dead organism, or an in vitro controlled environment. It can include animal testing and clinical trials. It can include other animals and living organism

The term “ex vivo” according to the invention can be any type of any experimentation or measurements done in or on tissue or isolated primary cells in an artificial environment outside the organism e.g. with the minimum alteration of natural conditions. It can include cell free expression systems, cell lines and isolated cells.

The PS-DNA according to this invention can be of any length, sequence and it can be single strand as well as double strand. It can have an iced combination of PS-DNA with other small molecules. It can be modified for example methylated or labeled with a fluorescent tag.

Eukaryotic cell according to the invention can be any organism whose cells contain complex structures inside the membranes. The defining membrane-bound structure that sets eukaryotic cells apart from prokaryotic cells is the nucleus, or nuclear envelope. It can be from any type of cell or specie.

The stem cell according to this invention can be any cell that has the ability to renew them selves through mitotic cell division and differentiate into a diverse range of specialized cell types. For example it can be embryonic stem cells that are isolated from the inner cell mass of blastocysts, and adult stem cells that are found in adult tissues. Moreover it can contain Stem cells that were grown and transformed into specialized cells. It can include stem cells with different differentiation potential (the potential to differentiate into different cell types) for example: Totipotent (a.k.a omnipotent) stem cells can differentiate into embryonic and extraembryonic cell types. Such cells can construct a complete, viable, organism. These cells are produced from the fusion of an egg and sperm cell. Cells produced by the first few divisions of the fertilized egg are also totipotent. Pluripotent stem cells are the descendants of totipotent cells and can differentiate into nearly all cells i.e. cells derived from any of the three germ layers.

The natural membrane protein according to this invention can be any membrane protein (see above). It can include a neural protein in the central nerve system or peripheral system. It can be a protein of any animal specie and in any tissue.

The vector according to this invention can be any DNA molecule used as a vehicle to transfer foreign genetic material into another cell. It can include bacteriophages and other viruses, cosmids, and artificial chromosomes. Common to all engineered vectors are an origin of replication, a multicloning site, and a selectable marker. It can contain and insertion and can include many promoters.

The fluorescent protein according to this invention can be any protein which exhibits emission of electromagnetic radiation light by a substance that has absorbed radiation of a different wavelength. In most cases, absorption of light of a certain wavelength induces the emission of light with a longer wavelength (and lower energy). It can be attached to any other molecule and can be modified.

The invention is based on the following findings.

It has been found according to the invention that innate, as well as transfected PrP, were degraded when exposed to PS-DNA in different cell types and various species. The degradation of PrP following PS-DNA exposure is time dependent, and when PS-DNA was removed from the media, PrP levels returned to normal. Post-translational modification of PrP did not affect this degradation since non-glycosilated, mono-glycosilated and di-glycosilated isoforms of PrP were all degraded following PS-DNA exposure. It was found according to the invention that when the N terminus of PrP (aa23-99) was deleted, PrP became resistant to PS-DNA degradation. Additionally, secreted PrP, PrP redirected to the mitochondria, Cytoplasm, or to the Nucleus, became resistant to PS-DNA degradation as well. Surprisingly, expression levels of proteins that are resistant to PS-DNA, such as Thy1, Dpl, GFP and CFP, became susceptible to PS-DNA degradation, following the introduction of PrP or the N-terminus of PrP into their sequence. The findings according to the invention pave the way to the modulation of membrane proteins. Attenuation of protein expression in a conditional and reversible way, solely on the protein level, can be exploited to modulate the expression of regulatory proteins. In one embodiment, modulation of membrane proteins can be exploited to elucidate the biological function of such proteins. The findings according to the invention suggest that PrP and its degradation following DNA exposure may have a physiological role in cell signaling of viral infection or of death of a neighboring cell.

Expression of a peptide or protein of the invention in a mammalian cell may be carried out by inserting the DNA coding for the protein or peptide into a vector comprising a promoter, optionally an intron sequence and splicing donor/acceptor signals, and further optionally comprising a termination sequence. These techniques are in general described in Ausubel et al., Current Protocols in Molecular Biology (Chapter 16), Greene Publications and Wiley Interscience, New York, N.Y., 1987-1995; Sambrook et al., Molecular Cloning: A Laboratory Manual, Cold Spring Harbor Laboratory, Cold Spring Harbor, N.Y., 1989.

The definition of “pharmaceutically acceptable” is meant to encompass any carrier, which does not interfere with effectiveness of the biological activity of the active ingredient and that is not toxic to the host to which it is administered. For example, for parenteral administration, the active protein(s) may be formulated in a unit dosage form for injection in vehicles such as saline, dextrose solution, serum albumin and Ringer's solution.

The active ingredients of the pharmaceutical composition according to the invention can be administered to an individual in a variety of ways. The routes of administration include intradermal, transdermal (e.g. in slow release formulations), intramuscular, intraperitoneal, intravenous, subcutaneous, oral, intracranial, epidural, topical, and intranasal routes. Any other therapeutically efficacious route of administration can be used, for example absorption through epithelial or endothelial tissues or by gene therapy wherein a DNA molecule encoding the active agent is administered to the patient (e.g. via a vector), which causes the active agent to be expressed and secreted in vivo. In addition, the polypeptide(s) according to the invention can be administered together with other components of biologically active agents such as pharmaceutically acceptable surfactants, excipients, carriers, diluents and vehicles.

The findings according to the invention reveal that PS-DNA degraded membrane PrP in different cell types derived from multiple species, indicating a basic biological phenomenon. It was found according to the invention PrP glycosilation was not essential for this degradation. Nevertheless, only anchored PrP was downregulated in the presence of PS-DNA. Using chimeras of PrP and its three-dimensional structure homologue, Dpl, we revealed that the N-terminus of PrP, or portions of it, is essential for this degradation. Lastly, attaching this domain and the GPI anchoring portion of PrP to other proteins, made these protein susceptible to PS-DNA.

Karpuj et al and Kocisko et al demonstrated that phosphoro thioate DNA (PS-DNA) can serve as a potent antiscrapie compound (Karpuj et al. 2007)(Kocisko et al. 2006). Karpuj et al expanded the phenomenon and revealed that PS-DNA degrades not only PrPSc but PrPC as well.

It has been found according to the invention that polyanion molecules such as PS-DNA can be used as a mediator for membrane protein levels in cell culture, similar to other protein knockout tools.

It was shown according to the invention that PS-DNA down-regulates native as well as trasnfected PrP, levels. Moreover, this phenomenon occurs in many cell types from various species (FIG. 1).

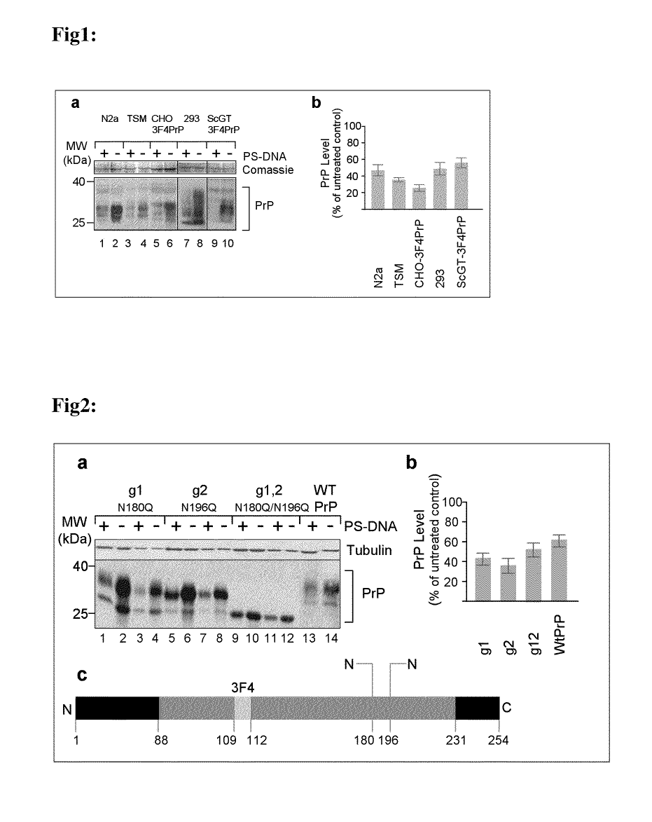

The initial evidence indicating that the N-terminus might be the essential portion of PrP, for its down-regulation following PS-DNA exposure, resided from the findings that the glycosilation at the c-terminus of PrP, was not essential and did not interfere with the ability of PS-DNA to down-regulate PrP levels (FIG. 2). Additionally, non-glycosilated PrP is as susceptible to PS-DNA as the other PrP glycoforms.

Testing the susceptibility or different deletion mutants to PS-DNA exposure verify that indeed deletion of the N-terminus of PrP aa 23-88 made PrP resistant to PS-DNA degradation while deletion of an essential area in PrP for prion infectivity at the C terminus (aa 141-176) was still susceptible (FIG. 4). This discovery was reinforced when other proteins that are resistant to PS-DNA exposure became susceptible when they were tagged only with the N-terminus of PrP. Of note, Calzolai et al demonstrated that epitope aa 26-41 contains a conserved PrP sequence among different vertebrates with no three dimensional structure homologies. Without being bound by the mechanism, that indicates that this area may provides an essential function of PrP which is conserved in evolution and the degradation of PrP to PS-DNA might elucidate this function.

Unexpectedly, when looking into the essential epitope within the N-terminus it was found according to the invention that regions of either side of aa 23-88 at the N-terminal sites present in mutant #2, #3 and #1 such as aa 51-88, aa 23-68 and 23-48aa, respectively were enough to reverse the resistance created by the elimination of this entire epitope (FIG. 5).

The N-terminus of PrP epitope (aa 51 to 88) contains the copper binding octarepeat peptide domain (Pauly & Harris 1998)(Rachidi 2002)(Stockel et al. 1998) besides it's DNA binding (Lima et al. 2006)(Cordeiro et al. 2001). When swapping mouse PrP aa23-90 with the homologous epitope from Xenopus Laevis PrP aa 23-69 which lucks the copper binding domain, this construct is even more susceptible to PS-DNA degradation compared to control (FIG. 2). Interestingly, this result correlates with the increase in PrP degradation observed in the construct omitted from aa 68-92 (FIG. 4, construct #3; 37% +/−2.2% reduction, compared to #1; 65% +/−8.7% reduction, respectively). It is known that this domain binds Copper (Hornshaw 1995, Brown 1997, Stockel 1998, Viles 1999)(Rachidi 2002). Without being bound by the mechanism, this might indicate that the copper-binding domain might directly interfere with PS-DNA binding, or that the structure of the N-terminus changes in the absence of this binding domain leading to more exposure to the PS-DNA binding. Another possibility is that the attraction of copper molecules to PrP around the DNA binding domain can inhibit the PS-DNA effect. Moreover, it is possible by using the approaches of the present invention to create further mutants within the N-terminus of PrP or chimeras in order to find the minimal essential PrP-tag for PS-DNA mediated degradations.

Shyang et al indicated that polyamines lead to increase internalization of PrP (Shyng et al. 1995). The same phenomenon was attributed to PS-DNA (Kocisko et al. 2006). Therefore differences in internalization may be considered. It was found according to the invention that cells that were not treated with PIPLC and treated with PS-DNA showed significant reduction of PrP levels (FIG. 8 lane 2 and 4 respectively), and there was almost no PrP secreted to the medium (FIG. 8 lanes 6 and 8 respectively). Moreover, there was no residual PrP in the cells that were treated with PS-DNA and treated with PIPLC, indicating that PrP within the cell was mostly degraded as well (FIG. 8 lanes 1). Therefore these sets of experiments provide the first evidence that PS-DNA might lead ultimately to a significant total degradation of the protein within the cells.

Previously it was shown that this degradation is reversible when ceasing treatment with PS-DNA for two weeks (Karpuj et al. 2007). The findings according to the invention demonstrate that down-regulation of PrP levels can be observed already after 4 hours of treatment with PS-DNA and recovery can be observed within the same frame time following removal of PS-DNA from the cell supernatant (FIG. 5). Nandi et al proposed that DNA induces partial unfolding of prion protein (P. K. Nandi et al. 2002). Without being bound by the mechanism, it is possible that the binding of PrP to DNA makes the entire protein more accessible to degradation or it is possible that DNA leads directly in some way to the degradation of the protein.

It was demonstrated that XenPrP shares the same biochemical properties as PrP but it is internalized faster (Nunziante et al. 2003). Nevertheless, the findings according to the invention demonstrate that XenPrP was one of the most susceptible constructs to PS-DNA degradation. This particularly interesting since it has been demonstrated in this study that PrP should be on the cell surface in order to be degraded by PS-DNA.

Kocisko et al demonstrated that PS-DNA colocalizes with membrane PrP enhancing PS-DNA internalization with PrP in cell cultures (Kocisko et al. 2006). Interestingly, Nunziante et al demonstrated that PrP with deletion of aa 48-92 is significantly more internalized than PrP construct were aa 23-51 are removed. Nevertheless, the results according to the invention indicate that the latter is more susceptible to PS-DNA. These results suggest that other factors are affecting this phenomenon besides differences in internalization rate. Moreover, the epitope that was demonstrated to be important for PrP endocytosis aa 23-31 KKRPKP is absence in construct #2 and it is nevertheless susceptible to PS-DNA degradation. This indicates that probably PS-DNA binds to another epitope on PrP or that the degradation of PrP through PS-DNA has a separate mechanism to that of internalization.

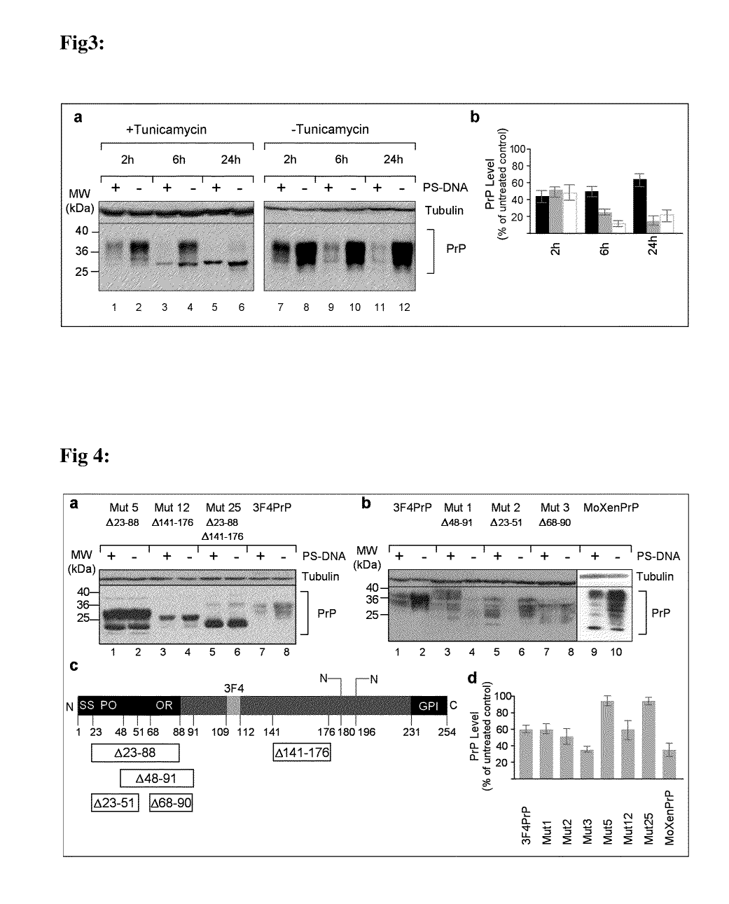

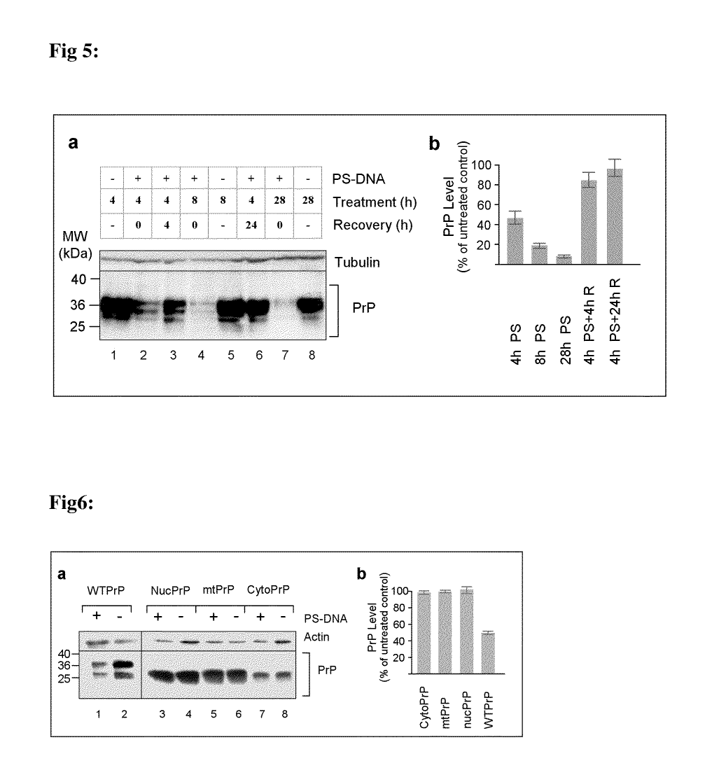

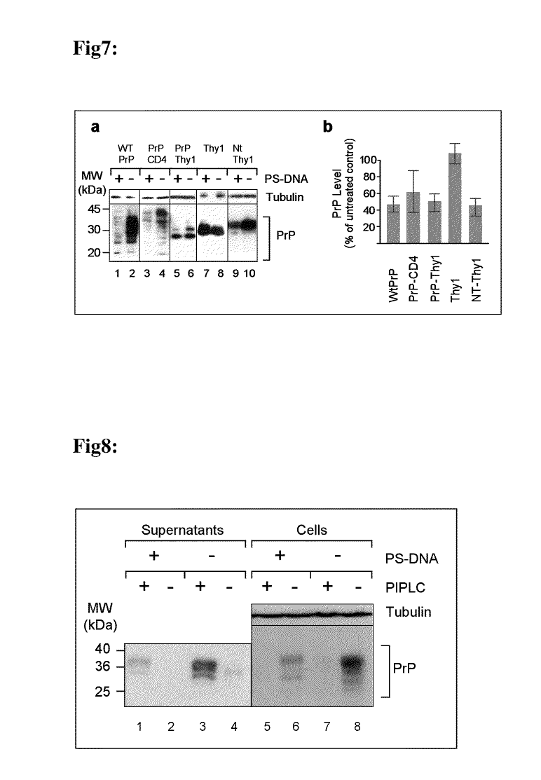

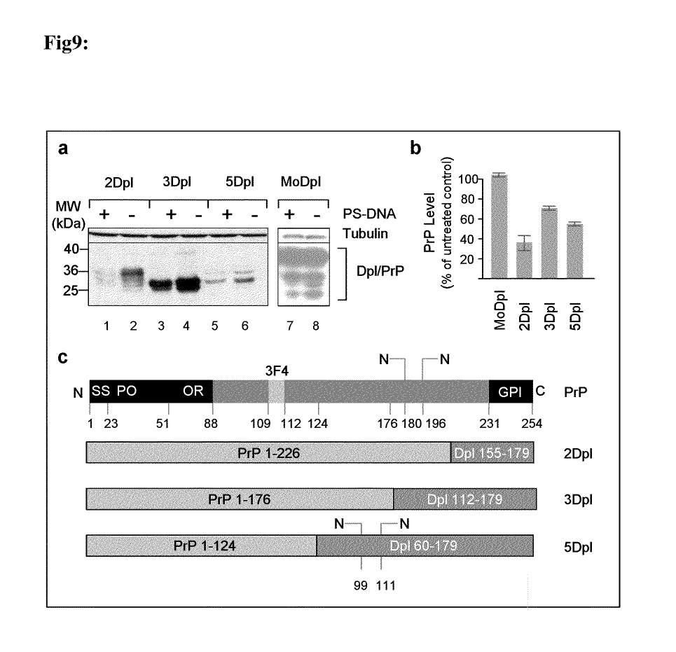

In addition to PrP N-terminus requirement for this degradation, it was demonstrated according to the invention that the protein should be anchored to the membrane of the cell (FIGS. 6, 7, and 8). It was found according to the invention that PrP redirected to the cytosole, Mitochondria or Nucleus became resistant to degradation following PS-DNA exposure (FIG. 6). One might think though that the fact that these three constructs are not glycosilated would contribute to their resistant. This conclusion is unlikely since the findings according to the invention demonstrate that non-glycosilated PrP is degraded in the presence of PrP (FIGS. 2 and 3). The results show that the only reason that these constructs are not degraded in the presence of PS-DNA is because of their location in the cell and not due to the fact that they are unglycosilated. PrP-CD4, which contains a heterologous C-terminal transmembrane domain instead of the GPI anchor (Taraboulos et al. 1995), and PrP with the GPI domain of Thy1 , both were down-regulated after exposure to PS-DNA. Regardless of the fact that PrP-CD4 targets proteins to clathrin-coated pits (Keller et al. 1992), while PrP is known to be targeted to caveolae-like domains both constructs were degraded by PS-DNA. Without being bound by the mechanism, this indicates that the anchoring to the membrane does not require a particular form of anchoring or any need for a specific membrane microenvironment.

In one embodiment of the invention modulation of the target protein of interest is carried out by using a binding molecule, capable of recognizing the target protein, tagged to PrP or fragments of it in combination with polyanions. The binding molecule can be e.g. a specific antibody a protein or a small molecule capable of recognizing the protein of interest. The binding molecule can be a ligand of the protein of interest. For example, if the protein of interest is Tumor Necrosis Factor (TNF) e.g. TRAIL, the molecule can be the TRAIL receptor-binding protein (Tanaka et al., 2009). Another example is Wnt. It is known that proteins belonging to the Wnt family mediate embryonic development, cell differentiation, proliferation and polarity. They do so through binding to cell surface molecules such as LRP5/6 and Frizzled. Thus a Wnt tagged to PrP is capable to bind Frizzled or LRP5/6 through Wnt and in the presence of a polyanion down-regulation of these receptors will lead to modulation of embryonic development, or cell differentiation, proliferation and polarity (Tanaka et al., 2009). The PrP tag can be the whole PrP or fragments thereof.

Without being bound by the mechanism, these sets of experiments indicate that PrP degradation is probably due to the interaction mainly with extracellular DNA. Another support to this hypothesis comes from Kociscko and his colleague. They have demonstrated that PrP co localizes with PS-DNA mostly at the cell surface and their co localization is not as prominent within the cell (Kocisko et al. 2006).

The finding according to the invention show that non-glycosilated PrP is susceptible to PS-DNA (FIG. 2) and lead us to use tunycamicine as one of the methods to test whether PS-DNA down-regulates levels of existing as well as newly synthesized PrP. Treatment with tunycamicin lead to accumulation of the non-glycosilated PrP representing the newly synthesized PrP (FIG. 3 lanes 2, 4, and 6). Densitometry analysis of the newly synthesized PrP in the presence or absence of PS-DNA revealed that newly synthesized PrP is significantly degraded compared to control (FIG. 3c black column; around 50% degradation) but its susceptibility did not change in time. Interestingly, existing PrP had the same degradation as newly synthesized PrP after 2 hours of exposure to PS-DNA. Nevertheless, Existing PrP showed significantly more susceptibility to in time (FIG. 3c green column). It is very possible that this is due to the fact that indeed in time, as long as tunycamicine is present in the medium and PrP is degraded naturally (FIG. 3c green column), there is less substrate to degrade and this drives the reaction faster towards degradation of PrP.

Tunicamycin induces ER-stress and activates unfolded Protein response, therefore translation of Proteins (and PrP) is inhibited while their degradation is enhanced (Hori & Elbein 1981). This might explain the fact that after a long treatment with tunicamycin (24 hours) the effect of PS-DNA is reduced (FIG. 3, green column). It is possible that Tunicamycin reduced cell surface expression of PrP and it is therefore less accessible to PS-DNA. In conclusion, PS-DNA degrades existing as well as newly synthesized PrP without affecting its ability to be synthesized de-novo upon the removal of PS-DNA.

So far we have tested the effect of PS-DNA on various proteins (N-CAM, tubulin, Thy1, GAPDH, and Dpl) and neither of them was degraded in the presence of PS-DNA (Karpuj et al. 2007)(FIGS. 2, 10, and 11). Our studies indicated that there is no specific PS-DNA sequence that is essential for PrP degradation. Nevertheless, the findings according to the invention show that degradation is very specific to PrP.

PrP has many different suggested biological functions. The properties of PrP as a DNA binding protein was previously demonstrated by others. This work though reveals that PrP is degraded in the presence of PS-DNA. Therefore it is possible that the degradation itself is providing a signal to the cells either of a necrotic event or viral infection (two pathways which are associated with significant levels of extra-cellular levels of nucleic acids).

It has been found according to the invention that only membrane anchored PrP (as opposed to nuclear, cytoplasmic or Mitochondrial PrP) is degraded in the presence of PS-DNA. Another possible way of looking at this observation is that this finding might imply that PrP expressed in different cell compartments might actually lead to different functions.

Surprisingly, it has been found according to the invention that various proteins, which in their native form are not affected by PS-DNA, became degraded when attached to full length PrP or only to a fragment of the N-terminus of PrP. Moreover, the degradation of these protein chimeras depended, exactly like in the case of wtPrP, on their anchoring to the membrane (FIGS. 10, and 11). These proteins did not have to share any homology to PrP and did not have to contain their original membrane anchoring. Also it was found according to the invention that the N terminus of PrP does not have to be attached to the N terminus of these proteins since N terminus of PrP tagged to the C terminus of CFP or YFP did not affect their ability to fluoresce (data not shown) but did down-regulate their levels in the presence of PS-DNA compared to controls (FIG. 12). The expected size on gel is 58.9 KDa and our experiment indicates a very similar result. Moreover, the fact that PrP with fluorescent tags has been degraded by PS-DNA, indicates that it is most likely that PrP in these chimeras has similar properties to the native PrP.

According to the invention, the transfected membrane protein tagged to PrP N-terminus, on either side of the protein, can be cloned into various vectors and still be degraded by PS-DNA (see material and methods for the variety of plasmid that were used in this study). These findings suggest a novel biological pathway of protein degradation. This pathway of protein degradation may be involved in the control of cell surface protein expression.

The invention provides a method in which proteins can be degraded in a controlled and transient way in cells. This tool has benefits to the scientific community compared to traditional knockout methods such as SiRNA. The findings according to the invention indicated that protein degradation is already observed within four hours of incubation with PS-DNA without the need of any transfection (FIG. 5). Advantageously, this method introduces few components to the system and makes the process easy. Moreover, this quick degradation is reversible, and removal of PS-DNA from the medium brings back PrP levels almost back to normal after 4 hours (80% of PrP compared to cells without PS-DNA exposure). Additionally, within the 4 hours and 8 hours of treatment with PS-DNA there is a gradual down-regulation of PrP. This will facilitate not just the exploration of protein function from “All to nothing” (as in the case of SiRNA when protein expression is Knocked out), but rather explore alterations in functional levels of the protein in question.

Nevertheless, the method according to the invention can be used to control the function of proteins in-vitro and also in-vivo. It is possible to use the method also as a diagnostic tool if the down-regulation of a certain protein leads to up-regulation to another protein. This ultimately will lead to the increase in desired signal detection.

It is well known that some DNA sequences can enhance the immune response or act as SiRNA. The fact that no specific sequence of PS-DNA is necessary to have this effect can be useful since sequences such as a polyG-22mer would probably not interfere indirectly with the expression of other proteins and would therefore only an effect on down-regulation of the desired tagged protein. With the advance of Gene therapy the chimeras and method according to the invention might become more relevant.

The chimeras and methods according to the invention can be used to knockout protein expression level in vivo and not just within the brain but in the different tissues.

As of today two major pathways of degradation have been described. The first of them is the pathway in which a protein leads to the cleavage or degradation of a stretch of nucleic acids as in the example of restriction enzymes which recognizes a specific region in a stretch of nucleic acids or as in the case of RNAses and DNAses, which lead to the compete degradation of stretches of nucleic acids. The second known pathway is one of a protein that leads to the partial cleavage or the total degradation of another protein as in the case of the proenzyme prothrombin or proteases like trypsin, respectively. This degradation can occur in the cytosol or in specific compartments such as the Lysosome, and the Proteasome. Previously we demonstrated that the final degradation of PrP in the presence of PS-DNA occurs at the lysosome and that PS-DNA has no effect on the transcription or translation of PrP. The results according to the invention extend to the degradation of other proteins and describes a novel pathway of degradation were the presence of nucleic acids, lead to the degradation of a protein. Another novel phenomenon in this pathway is that the specificity is unilateral, a specific sequence of the protein is needed while any PS-DNA can lead to this degradation-as long as the PS-DNA is longer than 15 mers (Karpuj et al 2007) and is in the sufficient concentration.

The chimeric protein and/or the method of the present invention can lead to, at least, two major practical contributions. As a result of the human genome project, new membrane proteins have been discovered though their biological function remains unknown. Creating chimeras of these membrane anchored proteins with the N terminal region of PrP. Then, transfecting them into cell lines which are depleted of these proteins, and exposing the cells to PS-DNA, can be used in order to attenuate the expression levels of specific proteins and by that allowing to unravel the pathways which these proteins are involved in and thus determine their basic function.

The invention provides ways of detecting PrP and co-localization with other molecules, proteins. Also, there is growing evidence that prion and other neurodegenerative disorders share pathways. Finding drugs using this chimera can help maybe curing not just prion disease but also other neurodegenerative disorders. In one embodiment, chimeras of PrP and other proteins are used with a fluorescent tag to measure the protein level of the tagged protein with PrP in a faster, cheaper reproducible, and easy way (and avoiding western blots).

As shown in the present invention, PrP can be tagged with protein epitopes using recombinant DNA techniques. For example all mouse PrP construct containing the hamster PrP epitope MKHM, recognized specifically by the monoclonal antibody 3F4 and differentiating the transfected PrP compared to native (FIGS. 4, 5, 10). Same phenomenon was shown for PrP bound to LAMP tag, or to a short region of Xenopus leavis PrP (FIG. 8), and for construct NT-Thy-1: chimera of aa 1-22 of PrP plus an NT flag, plus aa 23-88 of PrP plus Thy1 (FIG. 11). These tags could be placed in either side of PrP or within it and maintain biological properties of native PrP such as reaching the cell surface. As demonstrated in the findings according to the invention by the ability of degradation in the presence of PS-DNA which depends on the ability of the chimeric protein to reach the cell surface. Other examples for common tags used for this purpose are c-myc, HA, FLAG-tag, GST, VSV, HSV, V5, and 6×His. Tag Sequence HIS:HHHHHH, c-MYC:EQKLISEEDL, HA:YPYDVPDYA, VSV-G:YTDIEMNRLGK, HSV QPELAPEDPED, V5 GKPIPNPLLGLDST, and FLAG DYKDDDDK.

In one embodiment, chimeras with both cyan fluorescent protein (CFP) and yellow fluorescent protein (YFP) fused to PrP can be constructed and still the chimeric PrP is localized to the membrane. Moreover, the construct maintains the ability to fluoresce (data not shown), and PrP-YFP and PrP-CFP are still susceptible to PS-DNA degradation like the WT protein (FIG. 12). Moreover, it is shown according to the invention that when other proteins, like Dpl, were fused to PrP on its C-terminus side, this chimeric PrP was still localized in the cell surface and susceptible by PS-DNA (FIG. 10). Therefore, it is shown according to the invention, that it is possible with the chimeric proteins of the invention to create kits and methods for anti prion screen of drugs. In one embodiment of the invention, the method comprises using fluorescence resonance energy transfer (FRET) and these two constructs in combination or with one PrP construct with CFP and YFP in both ends. In one embodiment of the invention N2a cells are transfected in culture with the chimeric comprising two different fluorescent molecules, and the cells are seed in different wells. Small molecules are added before adding the infectious protein into the wells and FRET signal is checked. Since PrPc in the presence of the infectious protein changes its three dimensional structure into PrPsc the two fluorescent molecules on both ends of PrP will be closer to each other leading to a FRET signal. When a small molecule interferes with this process it can be expected a reduced FRET signal. This chimeric protein and its use allow detection of molecules that prevent Prion formation and would be good candidate molecule for early treatment of prion disease. In another embodiment, this entire process could be done by adding first the infectious material creating scrapie from the fluorescence tagged PrP and then add these small molecules. Molecules that will help reversing the prion formation could be potential drugs for prion disease. Since prion aggregates share some characteristics with other neurodegenerative disorder, some of these drugs could be tested in other neurodegenerative disorders. Moreover this method could be used for other proteins as well. Adding an additional tag (like the FLAG tag that will not interfere with the FRET or the PrP expression) could be useful to test the effect of these small molecules on total protein expression. In one embodiment the method provide molecules that will reduce the FRET signal but will not interfere with levels of protein expression.

CFP and YFP have the same sequence of GPF-green fluorescence protein but CFP has a mutation in Y66W and YFP has a mutation in T203Y (Klostermeier, Sears, Wong, Millar, & Williamson, 2004).

Karpuj et al and Kosikcso et al demonstrated that in vivo administration of PS-DNA might have a therapeutic application.

The chimera according to the invention can be used to create a transgenic mouse with fluorescent PrP and following infectivity and then PS-DNA application in vivo. In one embodiment of the invention, a chimeric PrP with the fluorescent tags containing a third tag such as HA plus the ability to redirect them into different organs is used. Thereafter, these proteins are isolated from the different cell types using the additional tag such as the HA tag and the FRET signaling is tested. Less FRET signaling demonstrates areas with less infectivity. The chimera helps to identify the optimal drug localization/delivery.

Studies in neurodegenerative disorder are providing more evidence that some of the proteins that are causing amyloid might share the same mechanism as prion, meaning that a template of a misfolded protein leads to the accumulation of other normal proteins into the misfolded form.

It is known therefore that this equilibrium is occurring:

PrPc+PrPsc<- - - >PrPsc+PrPsc

The more PrPc that is synthesized the more PrPsc that is created. It is possible that this is the case in other neurodegenerative disorders (Steelman et al., 2011).

It is also known that antibodies against PrP or Aβ are active in vivo and in vitro since they bind to PrP and shift this equation towards the unfolding of PrPsc towards PrPc. Therefore it might be possible that down-regulating PRPc in vivo by PS-DNA might reverse this equilibrium. If PrPc is involved in other disease the PS-DNA can be involved in curing them. Moreover it is possible that PS-DNA might down-regulate expression of other proteins involved in other diseases and PS-DNA could be used as a treatment.

PrP and homologues areas from several species which can be used according to the invention can be found in Cazolai et al. 2005.

Since the sequence of the PS-DNA is irrelevant, as shown by Karpuj et al and since PrP knock out mice are viable, this transient conditional degradation according to the invention can be used in the future for the regulation of expression of different proteins or pathways which can be regulated only in the presence of the PS-DNA without interfering with other major pathways. For instance, one major pitfall of stem cells at the moment is the ability to regulate their division and their activity after transplantation into the host. Administration of stem cells can be regulated, for instance, if these cells contain a regulating protein, which is fused to the N-terminus of PrP. In such case, the activity of these cells in the host could be regulated via PS-DNA administration. This will also prevent the repeated intrusive invasions of stem cell transplantations in patients.

Furthermore, if this phenomenon can be extended to studies in animal models, this might be a possible way to down-regulate levels of membrane anchored proteins, known to be involved in different diseases.

A “therapeutically effective amount” is such that when administered, the said peptides, DNA or virus of the invention induces a beneficial effect in preventing or the course of a disease, disorder or condition. The dosage administered, as single or multiple doses, to an individual may vary depending upon a variety of factors, including the route of administration, patient conditions and characteristics (sex, age, body weight, health, and size), extent of symptoms, concurrent treatments, frequency of treatment and the effect desired.

All references cited herein, including journal articles or abstracts, published or unpublished U.S. or foreign patent application, issued U.S. or foreign patents or any other references, are entirely incorporated by reference herein, including all data, tables, figures and text presented in the cited references. Additionally, the entire contents of the references cited within the references cited herein are also entirely incorporated by reference.

Reference to known method steps, conventional methods steps, known methods or conventional methods is not any way an admission that any aspect, description or embodiment of the present invention is disclosed, taught or suggested in the relevant art.

Having now described the invention, it will be more readily understood by reference to the following examples that are provided by way of illustration and are not intended to be limiting of the present invention.

### EXAMPLES

### Material and Methods.

(i) Cell cultures and transfections. Murin Neuroblastoma cell lines (N2a), Chinese Hamster Ovary cells (CHO), Hypothalamic cell line infected with Scrapie and stably transfected with prion (ScGT1-MHM2), were kindly provided by Albert Taraboulos laboratory (Butler et al. 1988)(Karpuj et al. 2007)(Schatzl et al. 1997).

Human Embryonic Kidney cells (HEK293) were purchased from ATCC.

Cells were grown in 6-cm dishes in Dulbecco's modified Eagle'd medium (DMEM) until attaining 90% to 95% confluence (Sigma Chemicals, MO, USA). Cells were trypsinized (Sigma Chemicals, MO, USA), and diluted ten-fold into 60-mm plates containing 4 mL of DMEM. On the following day, the cells were washed once with fresh DMEM and 2.5 mL of new medium was applied to the cells. Oligonucleotides were then added to the dish for variable periods of time. All incubations were performed at 37 C. Cells were harvested and maintained as described in Karpuj et al (Karpuj et al. 2007). Cell lines were grown in DMEM medium enriched with 10% v/v FBS (Kibbutz. Beth Ha'Emek, Israel), 1% v/v L-glutamine (Kibbutz. Beth Ha'Emek, Israel) and 1% v/v Penicillin-Sterptomycin (Sigma Chemicals, MO, USA); ScGT1 cells were grown in 50% v/v Optimem medium (Invitrogen-Gibco, USA), 43.5% DMEM, 5% v/v FCS, 0.5 v/v L-Glutamine and 1% v/v Penicillin-Sterptomycin. The cells were grown in 37° C., humidity atmosphere which contain 5% CO2. Following treatment cells on a 60-mm plates were incubated with 100 ml lysis buffer (10 mM Tris-HCl, pH 8; 100 mM NaCl; 0.5% NP-40; and 0.5% deoxycholate) for 5 min on ice and DNA aggregates were collected from the lysate using a sterile tip.

For transient transfection of the cells 90% to 95% confluence 10-cm dish was split into a 6 well plate in a dilution of 1:16. When reaching confluence of 50% to 60% cells were washed with PBS, and the medium was exchanged to 2 ml medium with FCS. Fugene reagent kit was used adding 2 mg of each construct per transfection. The following day the medium was exchanged to a fresh medium, and 72 hours post transfection cells were exposed to PS-DNA for 24 hours before cell lysis. For Stable transfections, 72 hours post transfection cells were exposed to selective medium containing 1 gr/L G418. N2a cells were used to transfect with the vector without any insert as a negative control for the transfection and positive control for selectivity of G418. Indeed, N2a cells transfected with the vehicle without insertion died after approximately 10 days whereas cells expressing the different constructs proliferated. After a period of three to four weeks stably transfected cells were established.

(ii) Treatment of Cells with Oligonucleotides. Oligonucleotides were purchased from TrilLink Biotechnologies (San Diego, Calif., USA) after HPLC purification and verification using mass spectroscopy. The oligonucleotides synthesized and used for the present study had the following base sequences:

CPG 22-mer CpG-PS-DNA: TGACTGTGAACGTTCGAGTGA
Scr 22-mer SCR-PS-DNA: CAGTGATAGCTATGTGAGCTAG

Both oligonucleotides were tested in all cell types and gave identical results to the one described in figure one (data not shown). We therefore used in following experiments either of these two oligonucleotides.

(iii) Constructs, transformation, and primers. pSPDX-contained resistant to Ampyciline and G418. Constructs g1, g2, g12, #5, and #25 were cloned into this vector. Resistant to Ampyciline provided positive selection of transformation. Resistance to G418 provided positive selection of transfection and established stable transfected cell lines. These constructs were kindly provided by Professor Stanly Prusiner.

pcDNA3.1—Contains resistance to Ampyciline and G418. Constructs #1, #2, #3, #5, NT-Thy1, PrP-Thy1, PrP-Lamp, MoXenPrP, and Thy1 were cloned into this vector. This clone was purchased from Invitrogen, USA

gWiz—Contains resistance to Kanamycine. Resistant to Kanamycine provided positive selection of transformation. This plasmid has high transformation and transfection yield and therefore was used for transient transfections. This clone was purchased from Aldevron, USA

pWE3—Contains resistance to Ampyciline and G418. Constructs 2Dpl, 3Dpl, and 5Dpl were cloned into this vector. These constructs were kindly provided by Professor Stanly prusiner.

In order to verify the inserts in each plasmid all constructs were sequenced using these two sets of primers (Sigma, Israel):

Mo-PrP-F: ATGGCGAACCTTGGCTACT
Mo-PrP-R: TTCCTGATCGTGGGATGA

The primers that were used to sub-clone PrP constructs #5, and #25 from pSPDX into gWiz are (Sigma, Israel):

NotI-MHM2-F

(contains the restriction site of Not1 and DNA sequence coding aa 1 to 6 in PrP MHM2:1-6MHM2): CTCTGCGGCCGCATGGCGAACCTTGGCTAC

BamHI-MHM2-R

(contains the restriction site of BamH1 and DNA sequence coding aa 249 to 255 in PrP MHM2: 249-255MHM2):

CTCTGGATCCTCATCCCACGATCAGGAA

The constructs within pSPDX were amplified using PCR reaction in the presence of these primers sets. Restriction enzymes NotI and BamHI were used in order to sub-clone the PCR inserts into gWiz.

Escherichia coli (DH10B) were kindly provided by Dr. Shaul Buderman. These bacteria were used to amplify the different PrP constructs. Selection for transformation was achieved exposing the DH10B to 50 mg/ml Ampyciline or Kanamicine depending on the DNA construct that was transformed.

This is the DNA sequence of PrP MHM2 cDNA:

ATGGCGAACCTTGGCTACTGGCTGCTGGCCCTCTTTGTGACTATGTGGA
CTGATGTCGGCCTCTGCAAAAAGCGGCCAAAGCCTGGAGGGTGGAACAC
CGGTGGAAGCCGGTATCCCGGGCAGGGAAGCCCTGGAGGCAACCGTTAC
CCACCTCAGGGTGGCACCTGGGGGCAGCCCCACGGTGGTGGCTGGGGAC
AACCCCATGGGGGCAGCTGGGGACAACCTCATGGTGGTAGTTGGGGTCA
GCCCCATGGCGGTGGATGGGGCCAAGGAGGGGGTACCCACAATCAGTGG
AACAAGCCCAGTAAGCCAAAAACCAACATGAAGCACATGGCCGGCGCCG
CGGCAGCTGGGGCCGTGGTGGGGGGCCTTGGTGGCTACATGCTGGGGAG
TGCCATGTCTAGACCCATGATCCATTTTGGCAACGACTGGGAGGACCGC
TACTACCGTGAAAACATGTACCGCTACCCTAACCAAGTGTACTACAGGC
CAGTGGATCAGTACAGCAACCAGAACAACTTCGTGCACGACTGCGTCAA
TATCACCATCAAGCAGCACACGGTCACCACCACCACCAAGGGGGAGAAC
TTCACCGAGACCGATGTGAAGATGATGGAGCGCGTGGTGGAGCAGATGT
GCGTCACCCAGTACCAGAAGGAGTCCCAGGCCTATTACGACGGGAGAAG
ATCCAGCAGCACCGTGCTTTTCTCCTCCCCTCCTGTCATCCTCCTCATC
TCCTTCCTCATCTTCCTGATCGTGGGATGA

This is the Protein sequence of PrP MHM2.

XXX represent amino acids before the initiation of the Methionin PrP at position 3.

XXXMANLGYWLLALFVTMWTDVGLCKKRPKPGGWNTGGSRYPGQGSPG
GNRYPPQGGTWGQPHGGGWGQPHGGSWGQPHGGSWGQPHGGGWGQGG
GTHNQWNKPSKPKTNMKHMAGAAAAGAVVGGLGGYMLGSAMSRPMIHF
GNDWEDRYYRENMYRYPNQVYYRPVDQYSNQNNFVHDCVNITIKQHTVT
TTTKGENFTETDVKMMERVVEQMCVTQYQKESQAYYDGRRSSSTVLFSSP
PVILLISFLIFLIVG

This is the amino acid sequence of the PrP construct mutated in position 183 called in our study construct PrP g1. Mutation N183Q,=in this sequence according to our numerating it is in position 183.

XXXMANLGYWLLALFVTMWTDVGLCKKRPKPGGWNTGGSRYPGQGSPG
GNRYPPQGGTWGQPHGGGWGQPHGGSWGQPHGGSWGQPHGGGWGQGG
GTHNQWNKPSKPKTNMKHMAGAAAAGAVVGGLGGYMLGSAMSRPMIHF
GNDWEDRYYRENMYRYPNQVYYRPVDQYSNQNNFVHDCVQITIKQHTVT
TTTKGENFTETDVKMMERVVEQMCVTQYQKESQAYYDGRRSSSTVLFSSP
PVILLISFLIFLIVG

This is the amino acid sequence of the PrP construct mutated in position 199 called in our study construct PrP g2. Mutation N199Q,=in this sequence according to our numerating it is in position 199

XXXMANLGYWLLALFVTMWTDVGLCKKRPKPGGWNTGGSRYPGQGSPG
GNRYPPQGGTWGQPHGGGWGQPHGGSWGQPHGGSWGQPHGGGWGQGG
GTHNQWNKPSKPKTNMKHMAGAAAAGAVVGGLGGYMLGSAMSRPMIHF
GNDWEDRYYRENMYRYPNQVYYRPVDQYSNQNNFVHDCVNITIKQHTVT
TTTKGEQFTETDVKMMERVVEQMCVTQYQKESQAYYDGRRSSSTVLFSSP
PVILLISFLIFLIVG

This is the amino acid sequence of the PrP construct mutated in position 183 called in our study construct PrP g1 and mutated in position 199 called in our study construct PrP g2. Mutations N183Q, and N199Q

XXXMANLGYWLLALFVTMWTDVGLCKKRPKPGGWNTGGSRYPGQGSPG
GNRYPPQGGTWGQPHGGGWGQPHGGSWGQPHGGSWGQPHGGGWGQGG
GTHNQWNKPSKPKTNMKHMAGAAAAGAVVGGLGGYMLGSAMSRPMIHF
GNDWEDRYYRENMYRYPNQVYYRPVDQYSNQNNFVHDCVQITIKQHTVT
TTTKGEQFTETDVKMMERVVEQMCVTQYQKESQAYYDGRRSSSTVLFSSP
PVILLISFLIFLIVG

This is the amino acid sequence of the PrP construct where aa 23-88 were deleted. This is construct PrP #5 in this study

XXXMANLGYWLLALFVTMWTDVGGTHNQWNKPSKPKTNMKHMA
GAAAAGAVVGGLGGYMLGSAMSRPMIHFGNDWEDRYYRENMYRY
PNQVYYRPVDQYSNQNNFVHDCVNITIKQHTVTTTTKGENFTETDVK
MMERVVEQMCVTQYQKESQAYYDGRRSSSTVLFSSPPVILLISFLIFLI
VG

This is the amino acid sequence of the PrP construct where aa 141-176 were deleted. This is construct PrP #12 in this study

XXXMANLGYWLLALFVTMWTDVGLCKKRPKPGGWNTGGSRYPGQ
GSPGGNRYPPQGGTWGQPHGGGWGQPHGGSWGQPHGGSWGQPHG
GGWGQGGGTHNQWNKPSKPKTNMKHMAGAAAAGAVVGGLGGYM
LGSAMSRPMFVHDCVNITIKQHTVTTTTKGENFTETDVKMMERVVE
QMCVTQYQKESQAYYDGRRSSSTVLFSSPPVILLISFLIFLIVG

This is the amino acid sequence of the PrP construct where 23-88 and aa 141-176 were deleted. This is construct PrP #25 in this study

XXXMANLGYWLLALFVTMWTDVGGTHNQWNKPSKPKTNMKHMA
GAAAAGAVVGGLGGYMLGSAMSRPMFVHDCVNITIKQHTVTTTTKG
ENFTETDVKMMERVVEQMCVTQYQKESQAYYDGRRSSSTVLFSSPP
VILLISFLIFLIVG

This is the amino acid sequence of the PrP construct where 48-92 were deleted. This is construct PrP #1 in this study

XXXMANLGYWLLALFVTMWTDVGLCKKRPKPGGWNTGGSRYPGQ
GSPGGGTHNQWNKPSKPKTNMKHMAGAAAAGAVVGGLGGYMLGS
AMSRPMIHFGNDWEDRYYRENMYRYPNQVYYRPVDQYSNQNNFVH
DCVNITIKQHTVTTTTKGENFTETDVKMMERVVEQMCVTQYQKESQ
AYYDGRRSSSTVLFSSPPVILLISFLIFLIVG

This is the amino acid sequence of the PrP construct where 23-51 were deleted. This is construct PrP #2 in this study

XXXMANLGYWLLALFVTMWTDVYPPQGGTWGQPHGGGWGQPHG
GSWGQPHGGSWGQPHGGGWGQGGGTHNQWNKPSKPKTNMKHMA
GAAAAGAVVGGLGGYMLGSAMSRPMIHFGNDWEDRYYRENMYRY
PNQVYYRPVDQYSNQNNFVHDCVNITIKQHTVTTTTKGENFTETDVK
MMERVVEQMCVTQYQKESQAYYDGRRSSSTVLFSSPPVILLISFLIFLI
VG

This is the amino acid sequence of the PrP construct where 68-92 were deleted. This is construct PrP #3 in this study

XXXMANLGYWLLALFVTMWTDVGLCKKRPKPGGWNTGGSRYPGQ
GSPGGNRYPPQGGTWGQPHGGGWGGGTHNQWNKPSKPKTNMKHM
AGAAAAGAVVGGLGGYMLGSAMSRPMIHFGNDWEDRYYRENMYR
YPNQVYYRPVDQYSNQNNFVHDCVNITIKQHTVTTTTKGENFTE
TDVKMMERVVEQMCVTQYQKESQAYYDGRRSSSTVLFSSPPVIL
LISFLIFLIVG

This is the amino acid sequence of the PrP construct where mouse PrP aa1-22 was connected to Xen Leavis PrP aa 23-69 and that was connected to MHM2 PrP aa94-254. This is construct is called MoXenPrP in this study. Big letters are amino acids of PrP and small are Xenopus PrP.

XXXMANLGYWLLALFVTMWTDVkksgggksktggwntgsnrnpn
ypggypgntggswgqGGGTHNQWNKPSKPKTNMKHMAGAAAAGA
VVGGLGGYMLGSAMSRPMIHFGNDWEDRYYRENMYRYPNQVYYR
PVDQYSNQNNFVHDCVNITIKQHTVTTTTKGENFTETDVKMMER
VVEQMCVTQYQKESQAYYDGRRSSSTVLFSSPPVILLISFLIFL
IVG

This is the amino acid sequence of PrP-Dpl Chimeras=Mouse PrP MHM2 aa 1-226 plus Doppel aa 155-179 (Including Doppel GPI anchor). This construct is called 2Dpl in this study. Big letters are amino acids of PrP and small are amino acids of Doppel:

XXXMANLGYWLLALFVTMWTDVGLCKKRPKPGGWNTGGSR
YPGQGSPGGNRYPPQGGTWGQPHGGGWGQPHGGSWGQPHGGSWG
QPHGGGWGQGGGTHNQWNKPSKPKTNMKHMAGAAAAGAVVGGL
GGYMLGSAMSRPMIHFGNDWEDRYYRENMYRYPNQVYYRPVDQYS
NQNNFVHDCVNITIKQHTVTTTTKGENFTETDVKMMERVVEQMCVT
QYQKESQAYYDGR

[symbol]

aa translation:

XXXMANLGYWLLALFVTMWTDVGLCKKRPKPGGWNTGGSRYPGQGSPG
GNRYPPQGGTWGQPHGGGWGQPHGGSWGQPHGGSWGQPHGGGWGQGG
GTHNQWNKPSKPKTNMKHMAGAAAAGAVVGGLGGYMLGSAMSRPMIHF
GNDWEDRYYRENMYRYPNQVYYRPVDQYSNQNNFVHDCVNITIKQHTVT
TTTKGENFTETDVKMMERVVEQMCVTQYQKESQ

[symbol]

[symbol]

This is the amino acid sequence of PrP-Dpl Chimeras=Mouse PrP MHM2 aa 1-180 plus Doppel aa 112-179 (Including Doppel GPI anchor). This construct is called 3Dpl in this study. Big letters are amino acids of PrP and small are amino acids of Doppel:

XXXMANLGYWLLALFVTMWTDVGLCKKRPKPGGWNTGGSRYPGQ
GSPGGNRYPPQGGTWGQPHGGGWGQPHGGSWGQPHGGSWGQPHG
GGWGQGGGTHNQWNKPSKPKTNMKHMAGAAAAGAVVGGLGGYM
LGSAMSRPMIHFGNDWEDRYYRENMYRYPNQVYYRPVDQYSNQNN
FVHD

[symbol]

[symbol]

This is the amino acid sequence of PrP-Dpl Chimeras=Mouse PrP MHM2 aa 1-124 plus Doppel aa 60-179 (Including Doppel GPI anchor). This construct is called 5Dpl in this study. Big letters are amino acids of PrP and small are amino acids of Doppel:

XXXMANLGYWLLALFVTMWTDVGLCKKRPKPGGWNTGGSRYPGQ
GSPGGNRYPPQGGTWGQPHGGGWGQPHGGSWGQPHGGSWGQPHG
GGWGQGGGTIINQWNKPSKPKTNMKHMAGAAAAGAVV

[symbol]

[symbol]

[symbol]

[symbol]

This is the amino acid sequence of PrP-YFP chimera=Mouse PrP aa 1-23 plus the full length of YFP aa1670-3225 plus mouse MHM2 PrP aa24-254 PrP-CFP=Mouse PrP aa 1-23 plus the full length of CFP aa1670-3225 plus mouse MHM2 PrP aa24-254. Big letters represent PrP and small represent the fluorescent molecule:

XXXMANLGYWLLALFVTMWTDV

[symbol]

[symbol]

[symbol]

[symbol]

[symbol]

GLCKKRPKPGGWNTGGSRYPGQGSPGGNRYPPQGGTW
GQPHGGGWGQPHGGSWGQPHGGSWGQPHGGGWGQGGGTHNQWNKPSKPK
TNMKHMAGAAAAGAVVGGLGGYMLGSAMSRPMIHFGNDWEDRYYRENMY
RYPNQVYYRPVDQYSNQNNFVHDCVNITIKQHTVTTTTKGENFTETDVK
MMERVVEQMCVTQYQKESQAYYDGRRSSSTVLFSSPPVILLISFLIFLI
VG

These are the nucleic acid sequences, amino acid sequences, and the primers that were used to create the chimera of PrP and CFP. The same was done with YFP (The differ only in one position. For details see previous PrP-CFP detailed sequence). Underlined sequence: ggatcc represents the BamH1 sequence

ATGGCGAACCTTGGCTACTGGCTGCTGGCCCTCTTTGTGACTATGTGGACTGATGTCGGC
M A N L G Y W L L A L F V T M W T D V G
CTCTGCAAAAAGCGGCCAAAGCCTGGAGGGTGGAACACCGGTGGAAGCCGGTATCCCGGG
L C K K R P K P G G W N T G G S R Y P G

>Prp with BamHI

```
CTCTGCAAAAAGCGGCCAAAGCCTggatccGGAGGGTGGAACACCGGTGGAAGCCGGTATCCCGGG
L C K K R P K P G S G G W N T G G S R Y P G
>CFP
gatccATGGTGAGCAAGGGCGAGGAGCTGTTCACCGGGGTGGTGCCCATCCTGGTCGAGCTGGAC
G S M V S K G E E L F T G V V P I L V E L D
GGCGACGTAAACGGCCACAAGTTCAGCGTGTCCGGCGAGGGCGAGGGCGATGCCACCTAC
  G D V N G H K F S V S G E G E G D A T Y
GGCAAGCTGACCCTGAAGTTCATCTGCACCACCGGCAAGCTGCCCGTGCCCTGGCCCACC
  G K L T L K F I C T T G K L P V P W P T
CTCGTGACCACCCTGACCTGGGGCGTGCAGTGCTTCAGCCGCTACCCCGACCACATGAAG
  L V T T L T W G V Q C F S R Y P D H M K
CAGCACGACTTCTTCAAGTCCGCCATGCCCGAAGGCTACGTCCAGGAGCGCACCATCTTC
  Q H D F F K S A M P E G Y V Q E R T I F
TTCAAGGACGACGGCAACTACAAGACCCGCGCCGAGGTGAAGTTCGAGGGCGACACCCTG
  F K D D G N Y K T R A E V K F E G D T L
GTGAACCGCATCGAGCTGAAGGGCATCGACTTCAAGGAGGACGGCAACATCCTGGGGCAC
  V N R I E L K G I D F K E D G N I L G H
AAGCTGGAGTACAACTACATCAGCCACAACGTCTATATCACCGCCGACAAGCAGAAGAAC
  K L E Y N Y I S H N V Y I T A D K Q K N
GGCATCAAGGCCAACTTCAAGATCCGCCACAACATCGAGGACGGCAGCGTGCAGCTCGCC
  G I K A N F K I R H N I E D G S V Q L A
GACCACTACCAGCAGAACACCCCCATCGGCGACGGCCCCGTGCTGCTGCCCGACAACCAC
  D H Y Q Q N T P I G D G P V L L P D N H
TACCTGAGCACCCAGTCCGCCCTGAGCAAAGACCCCAACGAGAAGCGCGATCACATGGTC
  Y L S T Q S A L S K D P N E K R D H M V
CTGCTGGAGTTCGTGACCGCCGCCGGGATCACTCTCGGCATGGACGAGCTGTACAAGggatccTAA
  L L E F V T A A G I T L G M D E L K G S G -
>Prp
ATGGCGAACCTTGGCTACTGGCTGCTGGCCCTCTTTGTGACTATGTGGACTGATGTCGGC
  M A N L G Y W L L A L F V T M W T D V G
CTCTGCAAAAAGCGGCCAAAGCCTGGAGGGTGGAACACCGGTGGAAGCCGGTATCCCGGG
  L C K K R P K P G G W N T G G S R Y P G
CAGGGAAGCCCTGGAGGCAACCGTTACCCACCTCAGGGTGGCACCTGGGGGCAGCCCCAC
  Q G S P G G N R Y P P Q G G T W G Q P H
GGTGGTGGCTGGGGACAACCCCATGGGGGCAGCTGGGGACAACCTCATGGTGGTAGTTGG
  G G G W G Q P H G G S W G Q P H G G S W
GGTCAGCCCCATGGCGGTGGATGGGGCCAAGGAGGGGGTACCCACAATCAGTGGAACAAG
  G Q P H G G G W G Q G G G T H N Q W N K
CCCAGTAAGCCAAAAACCAACATGAAGCACATGGCCGGCGCCGCGGCAGCTGGGGCCGTG
  P S K P K T N M K H M A G A A A A G A V
GTGGGGGGCCTTGGTGGCTACATGCTGGGGAGTGCCATGTCTAGACCCATGATCCATTTT
  V G G L G G Y M L G S A M S R P M I H F
GGCAACGACTGGGAGGACCGCTACTACCGTGAAAACATGTACCGCTACCCTAACCAAGTG
  G N D W E D R Y Y R E N M Y R Y P N Q V
TACTACAGGCCAGTGGATCAGTACAGCAACCAGAACAACTTCGTGCACGACTGCGTCAAT
  Y Y R P V D Q Y S N Q N N F V H D C V N
ATCACCATCAAGCAGCACACGGTCACCACCACCACCAAGGGGGAGAACTTCACCGAGACC
  I T I K Q H T V T T T T K G E N F T E T
GATGTGAAGATGATGGAGCGCGTGGTGGAGCAGATGTGCGTCACCCAGTACCAGAAGGAG
  D V K M M E R V V E Q M C V T Q Y Q K E
TCCCAGGCCTATTACGACGGGAGAAGATCCAGCAGCACCGTGCTTTTCTCCTCCCCTCCT
  S Q A Y Y D G R R S S S T V L F S S P P
GTCATCCTCCTCATCTCCTTCCTCATCTTCCTGATCGTGGGATGA
  V I L L I S F L I F L I V G -
```

These are the two sets of primers that we have used in order to sequence our constructs both of the are underlined in the sequence of PrP above.

Prp-R
5′-AGGCTTTGGCCGCTTTTTGC-3′
Prp-F
5′-GGAGAAGATCCAGCAGCACC-3′

These is the order in which these chimeras were constructed:
PrPCD4=chimera of mouse MHM2 aa 1-230 plus TM domain CD4
PrP LAMP1=chimera of mouse PrP aa 1-230 plus LAMP1 aa127-162
PrP-Thy1=chimera of mouse MHM2 PrP aa 1-230 plus GPI Thy1 aa127-162

*These are the specific sequences of the epitopes mentioned in this study:

This is the sequence of amino acids aa96-106-HNQWNKPSKPK on mouse PrP recognized specifically by the antibody D13.

This is the sequence of aa 68-92 that was deleted from MHM2PrP in construct #3:QPHGGSWGQPHGGSWGQPHGGGWGQG

This is the sequence of aa 48-92 that was deleted from MHM2PrP in construct #1:

GNRYPPQGGTWGQPHGGGWGQPHGGSWGQPHGGSWGQPHGGGWGQG-

This is the signal sequence epitop of PrP numbered 1-23:

MANLGYWLLALFVTMWTDVG

This is the amino acid sequence of aa 141-176 that was deleted from MHM2PrP in construct #12 and #25:

IHFGNDWEDRYYRENMYRYPNQVYYRPVDQYSNQNN

This is the amino acid sequence in PrPMHM2 that is specifically recognized by Ab 3F4 and can distinguish transfected from native PrPr: MKHM

This is the amino acid sequence of the PrP GPI anchor aa231-254

RSSSTVLFSSPPVILLISFLIFLIVG

This is the entire sequence of PrP aa 1-230 PrP MHM2 which was used as a template for most chimeras (in bold is the MHM2 epitope):

XXXMANLGYWLLALFVTMWTDVGLCKKRPKPGGWNTGGSRYPGQGSPG
GNRYPPQGGTWGQPHGGGWGQPHGGSWGQPHGGSWGQPHGGGWGQGGGT
HNQWNKPSKPKTNMKHMAGAAAAGAVVGGLGGYMLGSAMSRPMIHFGND
WEDRYYRENMYRYPNQVYYRPVDQYSNQNNFVHDCVNITIKQHTVTTTT
KGENFTETDVKMMERVVEQMCVTQYQKESQAYYDGR

(iv) TCA precipitations. Supernatant (500 ml) were diluted with equal volume of 10% TCA solution and incubated for lhour on ice. Samples were centrifuge, using the 5417R bench-top eppendorf centrifuge, at 20,000 rpm for 15 min. Supernatant were gently removed and the pellet was washed with 1 ml of cold Acetone. This procedure was repeated three times. Samples were incubated for 15 min in the chemical hood in order to get rid of residual acetone. The pellet was resuspended in 50 ml lysis buffer diluted 1:4 in DDW.

(v) Antibodies and Immunoblots. Protein concentration in each sample was determined using Bradford reagent (Bio-Rad, CA, USA). Serial dilutions of 2 mg/ml stock were used as standard. Samples were tested using a 96 well plate using Ultramicroplate readerELX808 model from Bio-tex instrument. Tested samples (50 mg or 100 mg/well) were loaded onto 12% SDS-PAGE gel. As a positive control Prestained protein molecular weight marker was loaded onto the gel (Fermentas, USA).

FabD13—Recombinant antibody recognizes murine PrP epitope 96-106 diluted 1:1000 (In-pro, USA). Contains human Fab and can be recognized by the goat anti human Fab diluted 1:5000 (Pierce IL, USA).

3F4—monoclonal antibody recognizes methionine residues at position 112-109 in Hamster PrP (Sigma Chemicals, MO, USA). This antibody allows distinguishing between endogenous PrP and trasfected PrP constructs containing this Hamster epitope. 3F4 antibodies were diluted 1:2000. Tubulin antibody-monoclonal antibody recognizing murine and human alpha-Tubulin (Sigma Chemicals, MO, USA). This antibody was diluted 1:10,000. These primary monoclonal antibodies were recognized using a goat anti mouse IgG diluted 1:5000. Anti FLAG-Rabbit polyclonal antibody specifically recognizing amino acids DYKDDDDK tagging MoDpl and NT-Thy-1 (Sigma Chemicals, MO, USA). This antibody was diluted 1:5000 and recognized using a Donkey anti Rabbit antibody diluted 1:7500 (Amersham International Life Science, England)). Anti-HA-monoclonal antibody recognizes amino acids YPYDVPDYA in Thy-1 (Santa Cruz Biotechnology, Inc, USA). This antibody was diluted 1:1000. Sheep anti mouse IgG was used as a secondary antibody diluted 1:7500). All secondary antibodies were Peroxidase-conjugated, and diluted in TBST (TBS plus 0.5% V/V Tween 20) containing 5% skim-milk powder.

(vi) Tunicamycine treatment. Tunicamycine blocks N-glycosidic bonds by inhibiting the conversion of N-acetylglucosaminel-phosphate to dolichol monophosphte. Therefore, it inhibits PrP glycosilation. Tunicamycine was diluted in DMEM (1.5 mg/ml-stock solution). N2a Cells were grown in 6-cm dishes in DMEM until attaining 90% to 95% confluence. Cells were trypsinized, and diluted ten-fold into 6 well plates containing 4 mL of DMEM. On the following day, the cells were washed once with fresh DMEM and 2.5 mL of new medium was applied to the cells. Tunicamycin was added to the media (final concentration of 1.5 n/ml), and 10 μM Oligonucleotides were then added to the dish for variable periods of time. Cells without Tunycamicin and/or PS-DNA were included as negative controls.

(vii) PIPLC treatment. The enzyme PIPLC specifically claves GPI anchored proteins leading to their secretion into the media. PIPLC was diluted to a final concentration of 200M with BSA 0.05%, NaCl 144 mM, Tris HCl 10 mM, pH7.4. N2a Cells were grown in 10-cm dish in DMEM until attaining 90% to 95% confluence. Cells were trypsinized, and diluted fifty-fold into 6 well plates containing 4 mL of DMEM. On the following day, the cells were washed once with fresh DMEM and 2.5 mL of new medium was applied to the cells. Oligonucleotides were then added to the dish (10 μM) for 72 hours. Cells without exposure to PS-DNA were included as negative control. Cells were then washed three times with preheated PBS solution (37 C) to remove residual PS-DNA. Fresh medium without FBS in the presence or absence of PIPLC (200 mM) was added to all wells. Cells were returned to the incubator at 37 C and lysed after 6 hours.

(viii) Densitometry assay and statistics. Protein quantification on western blot analysis were done using NIH Image J. Protein signal was measured compared to background. In order to eliminate technical difference derived from sample loading, PrP signal was normalized to total protein, using commasie blue staining or Tublin signal. Excel and SPSS programs were used in order to present the statistical results. T-test was used when two groups were compared to each other. For comparisons involving more then two groups Post-Hoc LSD one-way ANOVA was used. Treatment were considered to be significant when p<0.05.

### EXAMPLE 1

### Native PrP as Well as Transfected PrP is Degraded by PS-DNA in Various Cell Types from Different Origin

To further study the PrP degradation following PS-DNA exposure, the next experiment aimed to find out whether this type of degradation occurs only on neuronal cells or can occur in other cell types and species.

As of today the phenomenon of native PrP degradation via PS-DNA at the protein level was demonstrated solely in two neuronal mouse cell lines (N2a and GT1). In order to explore the possibility of a broader phenomenon we exposed Chinese hamster ovarian cells transfected with MHM2 PrP (CHO-MHM2), mouse neurocortical neuroblasts cells (TSM), as well as human kidney 293 cells, to 10 mM of PS-DNA for 24 hours. Two different sequences of PS-DNA were used, CpG-PS-DNA and SCR-PS-DNA. Both sequences gave identical results (data not shown) verifying our initial discovery that the PS-DNA sequence does not effect PrP (Karpuj et al. 2007). Identical cell lines seeded in parallel, and exposed to media without PS-DNA, were used as negative control. N2a cells as wells as Srapie infected GT1 cells transfected with MHM2-PrP (ScGT1), were used as positive control for PrP degradation. The results show that PS-DNA did not effect total protein expression observed by commasie staining of total protein in each lane.

Surprisingly, the reduction of PrP was observed in all cell lines independent of their origin and cell type (FIG. 1). Moreover, transfected as well as native PrP where degraded upon exposure to PS-DNA.

### EXAMPLE 2

### Glycosilation is Not Essential for PrP Degradation via PS-DNA

The following experiment was carried out in order to determine whether PrP post-modifications such as glycosilation are essential for PrP degradation via PS-DNA. PrP is a Glycosilated protein and therefore is represented by mainly three distinguished bands on western blot analysis using an anti PrP Ab. The lower band represents the non-glycosilated specie, the middle band represents the mono-glycosilated specie, and the upper band represents the di-glycosilated specie of PrP. This is due to the fact that PrP has two glycosilation sites at position 186 and 190. To assess whether PrP glycosilation is essential for PrP degradation mediated by PS-DNA, we used three different PrP mouse constructs. In these constructs both, or each of these glycosilation sites, were mutated (kindly provided by Professor Prusiner). All constructs contained the hamster PrP epitope recognized specifically by the monoclonal antibody 3F4. The constructs were stably transfected into N2a cells and exposed for 48 hours to 10 μM PS-DNA. N2a cells were used as positive control for PrP degradation and the same cell lines without exposure to PS-DNA were used as negative control. Our results indicate that PrP glycosilation is not essential for PrP degradation, since all three glycoforms were susceptible to PS-DNA degradation. (FIG. 2). The construct containing the mutation in the glycosilation site at position 180 was reduced to 43% (+/−9.8%). The construct containing the mutation at the glycosilation site at position 196 was reduced to 41% (+/−12.4%). Whereas the construct containing both mutations was reduced to 68% (+/−7.2%). This down-regulation of PrP levels, following exposure to PS-DNA, was statistically significant in all constructs compare to control (p<0.01).

### EXAMPLE 3

### PS-DNA Degrades Existing as Well as Newly Synthesized PrP

An in indirect approach was carried out in order to distinguish existing from newly synthesized PrP using Tunicamycine. Tunicamycin, blocks all N-linked glycosylation which is a post translational modification (Hori & Elbein 1981). Moreover, the findings of the preceding Example that all PrP glycoforms, including the non glycosilated specie of PrP are degraded in the presence of PS-DNA it allows to look into the effect of PS-DNA on the newly synthesized non-glycosilated PrP (FIG. 2b construct g12). Therefore, cells were incubated with or without 1.5 mg/ml of Tunicamycine for 2, 6 and 24 hours (FIGS. 3a and 3b respectively). PS-DNA was then added to these cells at the same time were Tunicamycine was added. The results show that cells treated with Tunicamycin accumulate the non-glycosilated specie of PrP. Interestingly, newly synthesized PrP (FIG. 3b black bars) as well as existing PrP (FIG. 3b green bars) were affected in the presence of PS-DNA.

### EXAMPLE 4

### The N Terminus but not the C Terminus of PrP is Essential for its Degradation via PS-DNA

In view that no specificity on the PS-DNA side was found on the PrP-PS-DNA interaction (Karpuj et al. 2007), it was studied whether the specificity is imparted by a protein sequence in PrP. The next experiment was carried out in order to assess to assess if there is any specificity sequence of PrP that is essential for PrP degradation via PS-DNA. For this purpose various PrP deletions mutants were used and transfected into N2a cells. Interestingly, the results demonstrate that the N-terminus deletion, specifically residues 23-88, but not the C-terminus of PrP abolished the ability of PS-DNA to degrade PrP (FIG. 4b construct 5 and 25 compared to construct 12).

### EXAMPLE 5

### Only the Deletion of the Entire N Terminus was Able to Abolish the Degradation of PrP via PS-DNA

It was further explored whether within the N terminus of PrP there is a specific epitope which is essential for this degradation. For this purpose three different constructs containing different deletions within the N-terminus of PrP were designed. The constructs were compared to the full N terminal deletion, previously showing a significant inhibition of PrP degradation Via PS-DNA (FIG. 4). Surprisingly, partial deletions did not abolish the ability of PS-DNA to degrade PrP, while the entire deletion of the N-terminus region 23-88 did (FIG. 4). It is interesting to note that some deletions are more potent compared to the full length PrP. Interestingly it seems that PrP mutant #3 missing aa 68-92 (SEQ ID NO3) is more susceptible to degradation in the presence of PS-DNA, compared to the PrP mutant #1 missing aa 48-92 (SEQ ID Nol) as shown in FIG. 4b. construct #3; 37% +/−2.2% reduction, compared to #1; 65% +/−8.7% reduction, respectively and compared to the WT PrP.

### EXAMPLE 6

### PrP is Degraded by PS-DNA when Anchored to the Membrane

It was further explored whether there is a specific cell compartment localization of PrP which is essential for its degradation in the presence of PS-DNA. For this purpose PrP was targeted either into the cytoplasm, mitochondria or to the nucleus of N2a cells. As shown in FIG. 2 and FIG. 3 the pattern of wild type PrP on western blot are three bands. The results show that the three different constructs of PrP that do not get imported into the ER/Golgi but rather were expressed in the cytosol (cytoPrP), nucleus (nucPrP) or the mitochondria (mtPrP), show only one band at the exact size of unglycosylated PrP. Surprisingly, neither of these constructs were affected in the presence of PS-DNA. As control the same cells were transfected with wtPrP and treatment of PS-DNA decreased significantly PrP levels on the cell surface (FIG. 6).

Next, it was explored whether the type of membrane anchoring of PrP is important.

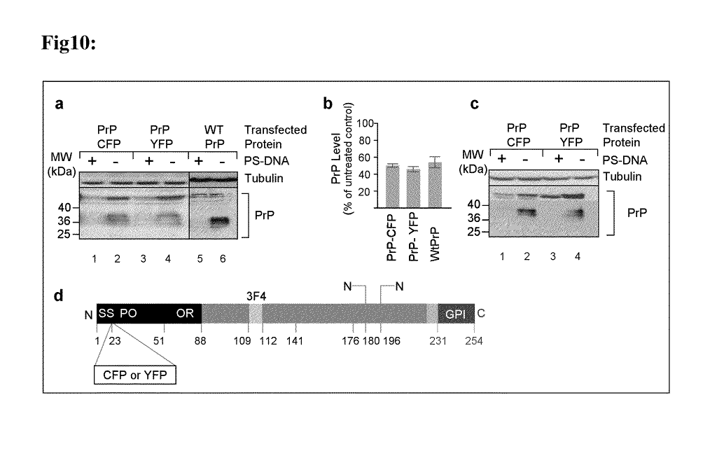

For this purpose in one construct, the GPI-anchor attachment signal (aa232-254) was replaced with a CD4 transmembrane domain. This replacement will allow anchoring the protein to the membrane but it will alter the membrane environment, shifting it from caveolae-like to a clathrin-coated pit-like topology and trafficking (Taraboulos et al 1995). Also, in another construct the GPI anchor of PrP was replaced to that of Thy1. The results show that both constructs were degraded by the PS-DNA. The results show that the way by which the protein is attached to the membrane or if it is caveolane like topology is not crucial for PrP degradation following exposure to PS-DNA. As a negative control we used a chimera of LAMP-PrP, which localizes PrP to the cytosol (FIG. 7).

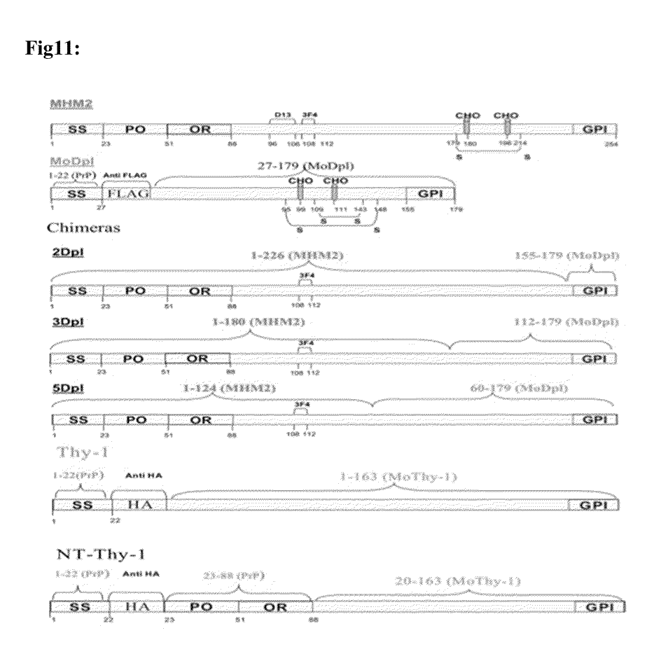

These experiments indicate that only the membrane PrP exposed to PS-DNA was degraded by the PS-DNA. In order to ascertain this finding in a different manner, cells were treated with 10 mM PS-DNA for 72 hours and thereafter exposed to 200 mM of PIPLC. PIPLC cleaves all GPI anchored membrane proteins and therefore will release membrane PrP to the supernatant. The results show that when cells were treated with PIPLC, after exposure to PS-DNA, there was less PrP secreted to the media indicating that membrane PrP was indeed degraded or possibly internalized into the cell in the presence of PS-DNA (FIG. 9 lane 5 and 7 respectively).

### EXAMPLE 7

### Attaching PrP at the N-Terminus of Other Proteins Leads to Their Degradation in the Presence of PS-DNA in Cell Culture

The possibility to degrade other membrane proteins that are resistant to PS-DNA degradation, by attaching them to the N-terminus of PrP was further explored. Resistant of Dpl to PS-DNA degradation was previously demonstrated (Karpuj et al. 2007). We therefore tested first chimeras of different portions of N-terminus PrP (Please specify which aa residues) with different portions of C-terminus of Dpl. Our results indicate that all the PrP-Dpl chimeras were degrade in the presence of PS-DNA (FIG. 10 construct 2Dpl, 3Dpl, and 5Dpl (2Dpl, PrP aa1-226 plus Dpl aa155-179; 3Dpl, PrP aal-180 plus Dpl aa 112-179; 5Dpl, PrP aal-124 plus Dpl aa60-179) while Dpl levels were not affected following PS-DNA exposure. Dpl and PrP are homologues in their there dimensional structure (Whyte et al. 2003). In order to verify if the degradation of these chimeras was due to the homology of these two proteins we built a chimera of N-terminal PrP (aa residues 1-88) with Thy1 which does not share a homologues structure with PrP. It is important to note that Thy1 is a GPI anchored membrane protein. Our experiments reveal that Thy1 became susceptible to PS-DNA degradation only when tagged to PtP N-terminus (FIG. 11). Thereafter, we inquired whether proteins that are not degraded by PS-DNA, and are not anchored to the cell surface in their native form, could be degraded in the presence of PS-DNA if tagged by the N terminus of PrP and anchored to the membrane. We then created a CFP construct attached to the signal sequence of PrP at their N-terminus while the PrP N-terminus and the GPI anchor of PrP were added to the C terminus of CFP. We then created the same construct with YFP. Interestingly, both CFP and YFP constructs were susceptible to PS-DNA degradation. This phenomenon was observed both in N2a cells and CHO cells (FIG. 10). We therefore conclude that proteins with no structural homology to PrP can be conditionally degraded by PS-DNA when tagged to N-terminus of PrP in either end of the protein as long as they are anchored to the cell membrane.

### REFERENCES

Atouf, F. et al., 1994. Tight Hormonal Control of PRP Gene Expression in Endocrine Pancreatic Cells. Biochemical and Biophysical Research Communications, 201(3), 1220-1226.Barret, A. et al., 2003. Evaluation of Quinacrine Treatment for Prion Diseases. J. Virol., 77(15), 8462-8469.
Brimacombe, D. B. et al., 1999. Characterization and polyanion-binding properties of purified recombinant prion protein. Biochemical Journal, 342(Pt 3), 605-613.
Bueler, H. et al., 1994. High prion and PrPSc levels but delayed onset of disease in scrapie-inoculated mice heterozygous for a disrupted PrP gene. Molecular Medicine, 1(1), 19-30.
Bileler, H. et al., 1992. Normal development and behaviour of mice lacking the neuronal cell-surface PrP protein. Nature, 356(6370), 577-582.
Butler, D. A. et al., 1988. Scrapie-infected murine neuroblastoma cells produce protease-resistant prion proteins. J. Virol., 62(5), 1558-1564.
Calzolai, L. et al., 2005. Prion protein NMR structures of chickens, turtles, and frogs. Proceedings of the National Academy of Sciences of the United States of America, 102(3), 651-655.
Carlson, G. A. et al., 1994. Prion isolate specified allotypic interactions between the cellular and scrapie prion proteins in congenic and transgenic mice. Proceedings of the National Academy of Sciences of the United States of America, 91(12), 5690-5694.
Collinge, J. et al., 1994. Prion protein is necessary for normal synaptic function. Nature, 370(6487), 295-297.
DeArmond, S. J. et al., 1987. Changes in the localization of brain prion proteins during scrapie infection. Neurology, 37(8), 1271.
Dunn, S. E., et al., 2006. Isoprenoids determine Th1/Th2 fate in pathogenic T cells, providing a mechanism of modulation of autoimmunity by atorvastatin. The Journal of Experimental Medicine, 203(2), 401-412.
Edenhofer, F. et al., 1996. Prion protein PrPc interacts with molecular chaperones of the Hsp60 family. J. Virol., 70(7), 4724-4728.
Gabus, C. et al., 2001. The Prion Protein Has RNA Binding and Chaperoning Properties Characteristic of Nucleocapsid Protein NCp7 of HIV-1. Journal of Biological Chemistry, 276(22), 19301-19309.
Hori, H. & Elbein, A. D., 1981. Tunicamycin Inhibits Protein Glycosylation in Suspension Cultured Soybean Cells. Plant Physiol., 67(5), 882-886.
Karpuj, M. V. et al., 2007. Phosphorothioate Oligonucleotides Reduce PrPSc Levels and Prion Infectivity in Cultured Cells. Molecular Medicine, 13(3-4), 190-198.
Kocisko, D. A. et al., 2006. Potent Antiscrapie Activities of Degenerate Phosphorothioate Oligonucleotides. Antimicrob. Agents Chemother., 50(3), 1034-1044.
Málaga-Trillo, E. et al., 2009. Regulation of Embryonic Cell Adhesion by the Prion Protein. PLoS Biol, 7(3), e1000055.
Manson J C et al., 1994. PrP gene dosage determines the timing but not the final intensity or distribution of lesions in scrapie pathology., 3(4), 331-340.
Miele, G. et al., 2002. Ablation of Cellular Prion Protein Expression Affects Mitochondrial Numbers and Morphology. Biochemical and Biophysical Research Communications, 291(2), 372-377.
Nunziante, M., Gilch, S. & Schätz, H. M., 2003. Essential Role of the Prion Protein N Terminus in Subcellular Trafficking and Half-life of Cellular Prion Protein. Journal of Biological Chemistry, 278(6), 3726-3734.
Prusiner, S. B. et al., 1993. Ablation of the prion protein (PrP) gene in mice prevents scrapie and facilitates production of anti-PrP antibodies. Proceedings of the National Academy of Sciences of the United States of America, 90(22), 10608-10612.
Rachidi, W., 2002. Expression of Prion Protein Increases Cellular Copper Binding and Antioxidant Enzyme Activities but Not Copper Delivery. Journal of Biological Chemistry, 278(11), 9064-9072.
Rieger, R. et al., 1997. The human 37-kDa laminin receptor precursor interacts with the prion protein in eukaryotic cells. Nat Med, 3(12), 1383-1388.
Rivera-Milla, E. et al., 2005. Disparate evolution of prion protein domains and the distinct origin of Doppel- and prion-related loci revealed by fish-to-mammal comparisons. FASEB J., 05-4279fje.
Schatzl, H. et al., 1997. A hypothalamic neuronal cell line persistently infected with scrapie prions exhibits apoptosis. J. Virol., 71(11), 8821-8831.
Schmitt-Ulms, G. et al., 2001. Binding of neural cell adhesion molecules (N-CAMs) to the cellular prion proteinl. Journal of Molecular Biology, 314(5), 1209-1225.
Shyng, S. et al., 1995. Sulfated Glycans Stimulate Endocytosis of the Cellular Isoform of the Prion Protein, PrP, in Cultured Cells. Journal of Biological Chemistry, 270(50), 30221-30229.
Steelman, L., et al., 2011. Roles of the Raf/MEK/ERK and PI3K/PTEN/Akt/mTOR pathways in controlling growth and sensitivity to therapy-implications for cancer and aging, Aging, 3(3), 192-222.
Stockel, J. et al., 1998. Prion Protein Selectively Binds Copper(II) Ions†. Biochemistry, 37(20), 7185-7193.
Tanaka, H. et al. 2009. PRMT5, a Novel TRAIL Receptor-Binding Protein, Inhibits TRAIL-Induced Apoptosis via Nuclear Factor-κB Activation. Molecular Cancer Research, 7(4), 557-569.
Vogtherr, M. et al., 2003. Antimalarial Drug Quinacrine Binds to C-Terminal Helix of Cellular Prion Protein. Journal of Medicinal Chemistry, 46(17), 3563-3564.
Vostal, J. G., Holada, K. & Simak, J., 2001. Expression of cellular prion protein on blood cells: Potential functions in cell physiology and pathophysiology of transmissible spongiform encephalopathy diseases. Transfusion Medicine Reviews, 15(4), 268-281.
Whyte, S. M. et al., 2003. Stability and conformational properties of doppel, a prion-like protein, and its single-disulphide mutant. Biochemical Journal, 373(Pt 2), 485-494.

1. A method for regulating the level of a membrane-anchored protein of interest in a cell comprising the steps of
A—i) Introducing in the cell a chimeric protein comprising the protein of interest and prion protein (PrP) or a fragment thereof; or
ii) Exposing the cell to a chimeric protein comprising a protein of interest-binding molecule and PrP or a fragment thereof, and
B—Exposing the cell to a polyanion.

2. The method according to claim 1, wherein the PrP fragment is the N-terminus of PrP or a fragment thereof.

3. The method according to claim 1, wherein the protein of interest-binding molecule is a specific antibody raised against the protein of interest.

4. The method according to claim 1, wherein the protein of interest is a natural membrane protein.

5. The method according to claim 1, wherein the polyanion is PS-DNA.

6. A chimeric protein comprising PrP or a fragment thereof fused to a molecule capable of binding a membrane-anchored protein of interest.

7. The chimeric protein according to claim 6, wherein the binding protein is an antibody specific to the protein of interest.

8. A method for treating or preventing a disease, disorder or condition caused or augmented by unregulated levels of a membrane-anchored protein of interest comprising administration to a subject in need of
A—i) A therapeutically effective amount of a chimeric protein comprising prion protein (PrP) or a fragment thereof and the protein of interest, the DNA encoding the chimeric protein, or the vector encoding the DNA, or ii) A therapeutically effective amount of a chimeric protein comprising a protein of interest binding molecule and PrP or a fragment thereof, the DNA encoding the chimeric protein, the vector encoding the DNA; and
B—Subsequent administration of a therapeutically effective amount of polyanion.

9. The method according to claim 8, wherein the binding molecule is a specific antibody raised against the protein of interest.


SEQUENCE LISTING (44 sequences)

SEQ ID NO: 1 - protein, 10 aa, Artificial sequence
  Synthetic construct
EQKLISEEDL

SEQ ID NO: 2 - protein, 9 aa, Artificial sequence
  Synthetic construct
YPYDVPDYA

SEQ ID NO: 3 - protein, 11 aa, Artificial sequence
  Synthetic construct
YTDIEMNRLGK

SEQ ID NO: 4 - protein, 11 aa, Artificial sequence
  Synthetic sequence
QPELAPEDPED

SEQ ID NO: 5 - protein, 14 aa, Artificial sequence
  Synthetic sequence
GKPIPNPLLGLDST

SEQ ID NO: 6 - protein, 8 aa, Artificial sequence
  Synthetic sequence
DYKDDDDK

SEQ ID NO: 7 - DNA, 21 bp, Artificial sequence
  Synthetic construct
tgactgtgaacgttcgagtga

SEQ ID NO: 8 - DNA, 22 bp, Artificial sequence
  Synthetic construct
cagtgatagctatgtgagctag

SEQ ID NO: 9 - DNA, 19 bp, Artificial sequence
  Synthetic construct
atggcgaaccttggctact

SEQ ID NO: 10 - DNA, 18 bp, Artificial sequence
  synthetic construct
ttcctgatcgtgggatga

SEQ ID NO: 11 - DNA, 30 bp, Artificial sequence
  synthetic construct
ctctgcggccgcatggcgaaccttggctac

SEQ ID NO: 12 - DNA, 28 bp, Artificial sequence
  Synthetic construct
ctctggatcctcatcccacgatcaggaa

SEQ ID NO: 13 - DNA, 765 bp, Mus Musculus
atggcgaaccttggctactggctgctggccctctttgtgactatgtggactgatgtcggcctctgcaaaaagcggccaaagcctggagggtggaacaccggtggaagccggtatcccgggcagggaagccctggaggcaaccgttacccacctcagggtggcacctgggggcagccccacggtggtggctggggacaaccccatgggggcagctggggacaacctcatggtggtagttggggtcagccccatggcggtggatggggccaaggagggggtacccacaatcagtggaacaagcccagtaagccaaaaaccaacatgaagcacatggccggcgccgcggcagctggggccgtggtggggggccttggtggctacatgctggggagtgccatgtctagacccatgatccattttggcaacgactgggaggaccgctactaccgtgaaaacatgtaccgctaccctaaccaagtgtactacaggccagtggatcagtacagcaaccagaacaacttcgtgcacgactgcgtcaatatcaccatcaagcagcacacggtcaccaccaccaccaagggggagaacttcaccgagaccgatgtgaagatgatggagcgcgtggtggagcagatgtgcgtcacccagtaccagaaggagtcccaggcctattacgacgggagaagatccagcagcaccgtgcttttctcctcccctcctgtcatcctcctcatctccttcctcatcttcctgatcgtgggatga

SEQ ID NO: 14 - protein, 254 aa, Mus Musculus
MANLGYWLLALFVTMWTDVGLCKKRPKPGGWNTGGSRYPGQGSPGGNRYPPQGGTWGQPHGGGWGQPHGGSWGQPHGGSWGQPHGGGWGQGGGTHNQWNKPSKPKTNMKHMAGAAAAGAVVGGLGGYMLGSAMSRPMIHFGNDWEDRYYRENMYRYPNQVYYRPVDQYSNQNNFVHDCVNITIKQHTVTTTTKGENFTETDVKMMERVVEQMCVTQYQKESQAYYDGRRSSSTVLFSSPPVILLISFLIFLIVG

SEQ ID NO: 15 - protein, 254 aa, Mus Musculus
MANLGYWLLALFVTMWTDVGLCKKRPKPGGWNTGGSRYPGQGSPGGNRYPPQGGTWGQPHGGGWGQPHGGSWGQPHGGSWGQPHGGGWGQGGGTHNQWNKPSKPKTNMKHMAGAAAAGAVVGGLGGYMLGSAMSRPMIHFGNDWEDRYYRENMYRYPNQVYYRPVDQYSNQNNFVHDCVQITIKQHTVTTTTKGENFTETDVKMMERVVEQMCVTQYQKESQAYYDGRRSSSTVLFSSPPVILLISFLIFLIVG

SEQ ID NO: 16 - protein, 254 aa, Mus Musculus
MANLGYWLLALFVTMWTDVGLCKKRPKPGGWNTGGSRYPGQGSPGGNRYPPQGGTWGQPHGGGWGQPHGGSWGQPHGGSWGQPHGGGWGQGGGTHNQWNKPSKPKTNMKHMAGAAAAGAVVGGLGGYMLGSAMSRPMIHFGNDWEDRYYRENMYRYPNQVYYRPVDQYSNQNNFVHDCVNITIKQHTVTTTTKGEQFTETDVKMMERVVEQMCVTQYQKESQAYYDGRRSSSTVLFSSPPVILLISFLIFLIVG

SEQ ID NO: 17 - protein, 254 aa, Mus Musculus
MANLGYWLLALFVTMWTDVGLCKKRPKPGGWNTGGSRYPGQGSPGGNRYPPQGGTWGQPHGGGWGQPHGGSWGQPHGGSWGQPHGGGWGQGGGTHNQWNKPSKPKTNMKHMAGAAAAGAVVGGLGGYMLGSAMSRPMIHFGNDWEDRYYRENMYRYPNQVYYRPVDQYSNQNNFVHDCVQITIKQHTVTTTTKGEQFTETDVKMMERVVEQMCVTQYQKESQAYYDGRRSSSTVLFSSPPVILLISFLIFLIVG

SEQ ID NO: 18 - protein, 182 aa, Mus Musculus
MANLGYWLLALFVTMWTDVGGTHNQWNKPSKPKTNMKHMAGAAAAGAVVGGLGGYMLGSAMSRPMIHFGNDWEDRYYRENMYRYPNQVYYRPVDQYSNQNNFVHDCVNITIKQHTVTTTTKGENFTETDVKMMERVVEQMCVTQYQKESQAYYDGRRSSSTVLFSSPPVILLISFLIFLIVG

SEQ ID NO: 19 - protein, 218 aa, Mus Musculus
MANLGYWLLALFVTMWTDVGLCKKRPKPGGWNTGGSRYPGQGSPGGNRYPPQGGTWGQPHGGGWGQPHGGSWGQPHGGSWGQPHGGGWGQGGGTHNQWNKPSKPKTNMKHMAGAAAAGAVVGGLGGYMLGSAMSRPMFVHDCVNITIKQHTVTTTTKGENFTETDVKMMERVVEQMCVTQYQKESQAYYDGRRSSSTVLFSSPPVILLISFLIFLIVG

SEQ ID NO: 20 - protein, 146 aa, Mus Musculus
MANLGYWLLALFVTMWTDVGGTHNQWNKPSKPKTNMKHMAGAAAAGAVVGGLGGYMLGSAMSRPMFVHDCVNITIKQHTVTTTTKGENFTETDVKMMERVVEQMCVTQYQKESQAYYDGRRSSSTVLFSSPPVILLISFLIFLIVG

SEQ ID NO: 21 - protein, 208 aa, Mus Musculus
MANLGYWLLALFVTMWTDVGLCKKRPKPGGWNTGGSRYPGQGSPGGGTHNQWNKPSKPKTNMKHMAGAAAAGAVVGGLGGYMLGSAMSRPMIHFGNDWEDRYYRENMYRYPNQVYYRPVDQYSNQNNFVHDCVNITIKQHTVTTTTKGENFTETDVKMMERVVEQMCVTQYQKESQAYYDGRRSSSTVLFSSPPVILLISFLIFLIVG

SEQ ID NO: 22 - protein, 225 aa, Mus Musculus
MANLGYWLLALFVTMWTDVYPPQGGTWGQPHGGGWGQPHGGSWGQPHGGSWGQPHGGGWGQGGGTHNQWNKPSKPKTNMKHMAGAAAAGAVVGGLGGYMLGSAMSRPMIHFGNDWEDRYYRENMYRYPNQVYYRPVDQYSNQNNFVHDCVNITIKQHTVTTTTKGENFTETDVKMMERVVEQMCVTQYQKESQAYYDGRRSSSTVLFSSPPVILLISFLIFLIVG

SEQ ID NO: 23 - protein, 227 aa, Mus Musculus
MANLGYWLLALFVTMWTDVGLCKKRPKPGGWNTGGSRYPGQGSPGGNRYPPQGGTWGQPHGGGWGGGTHNQWNKPSKPKTNMKHAGAAAAGAVVGGLGGYMLGSAMSRPMIHFGNDWEDRYYRENMYRYPNQVYYRPVDQYSNQNNFVHDCVNITIKQHTVTTTTKGENFTETDVKMMERVVEQMCVTQYQKESQAYYDGRRSSSTVLFSSPPVILLISFLIFLIVG

SEQ ID NO: 24 - protein, 220 aa, Artificial sequence
  Synthetic sequnec
MANLGYWLLALFVTMWTDVKKSGGGKSKTGGWNTGSNRNPNYPGGYPGNTGGSWGQGGGTHNQWNKPSKPKTNMKHMAGAAAAGAVVGGLGGYMLGSAMSRPMIHFGNDWEDRYYRENMYRYPNQVYYRPVDQYSNQNNFVHDCVNITIKQHTVTTTTKGENFTETDVKMMERVVEQMCVTQYQKESQAYYDGRRSSSTVLFSSPPVILLISFLIFLIVG

SEQ ID NO: 25 - protein, 254 aa, Mus Musculus
MANLGYWLLALFVTMWTDVGLCKKRPKPGGWNTGGSRYPGQGSPGGNRYPPQGGTWGQPHGGGWGQPHGGSWGQPHGGSWGQPHGGGWGQGGGTHNQWNKPSKPKTNMKHMAGAAAAGAVVGGLGGYMLGSAMSRPMIHFGNDWEDRYYRENMYRYPNQVYYRPVDQYSNQNNFVHDCVNITIKQHTVTTTTKGENFTETDVKMMERVVEQMCVTQYQKESQAYYDGRRGAALRVAVDQPAMVCLLGFVWFIVK

SEQ ID NO: 26 - protein, 237 aa, Mus Musculus
MANLGYWLLALFVTMWTDVGLCKKRPKPGGWNTGGSRYPGQGSPGGNRYPPQGGTWGQPHGGGWGQPHGGSWGQPHGGSWGQPHGGWGQGGGTHNQWNKPSKPKTNMKHMAGAAAAGAVVGGLGGYMLGSAMSRPMIHFGNDWEDRYYRENMYRYPNQVYYRPVDQYSNQNNFVHDNATQAANQAEFSREKQDSKLHQRVLWRLIKEFWLERGAALRVAVDQPAMVCLLGFVWFIVK

SEQ ID NO: 27 - protein, 242 aa, Mus Musculus
MANLGYWLLALFVTMWTDVGLCKKRPKPGGWNTGGSRYPGQGSPGGNRYPPQGGTWGQPHGGGWGQPHGGSWGQPHGGSWGQPHGGGWGQGGGTHNQWNKPSKPKTNMKHMAGAAAAGAVVAFIKQGRKLDIDFGAEGNRYYAANYWQFPDGIYYEGCSEANVTKEMLVTSCVNATQAANQAEFSREKQDSKLHQRVLWRLIKEICSAKHCDFWLERGAALRVAVDQPAMVCLLGFVWFIVK

SEQ ID NO: 28 - protein, 533 aa, Mus Musculus
MANLGYWLLALFVTMWTDVMSIQHFRVALIPFFAAFCLPVFAHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEALRSALPAGWFIADKSGAGERGSRGIIAALGPDGKPSRIVVIYTTGSQATMDERNRQIAEIGASLIKHWGLCKKRPKPGGWNTGGSRYPGQGSPGGNRYPPQGGTWGQPHGGGWGQPHGGSWGQPHGGSWGQPHGGGWGQGGGTHNQWNKPSKPKTNMKHMAGAAAAGAVVGGLGGYMLGSAMSRPMIHFGNDWEDRYYRENMYRYPNQVYYRPVDQYSNQNNFVHDCVNITIKQHTVTTTTKGENFTETDVKMMERVVEQMCVTQYQKESQAYYDGRRSSSTVLFSSPPVILLISFLIFLIVG

SEQ ID NO: 29 - DNA, 120 bp, Mus Musculus
atggcgaaccttggctactggctgctggccctctttgtgactatgtggactgatgtcggcctctgcaaaaagcggccaaagcctggagggtggaacaccggtggaagccggtatcccggg

SEQ ID NO: 30 - protein, 40 aa, Mus Musculus
MANLGYWLLALFVTMWTDVGLCKKRPKPGGWNTGGSRYPG

SEQ ID NO: 31 - DNA, 67 bp, Mus Musculus
ctctgcaaaaagcggccaaagcctggatccggagggtggaacaccggtggaagccggtatcccgggg

SEQ ID NO: 32 - protein, 22 aa, Mus Musculus
LCKKRPKPGSGGWNTGGSRYPG

SEQ ID NO: 33 - DNA, 732 bp, Artificial sequence
  Synthetic construct
ggatccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggatcctaa

SEQ ID NO: 34 - protein, 222 aa, Artificial sequence
  Synthetic construct
GSMVSKGEELFTGVVPILVELDGDVNGHKFSVSGEGEGDATYGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKANFKIRHNIEDGSVQLADHYQQNTPIGDGPVLLPDNHYLSTQSALSKDPNEKRDHMV

SEQ ID NO: 35 - DNA, 20 bp, Artificial sequence
  Synthetic construct
aggctttggccgctttttgc

SEQ ID NO: 36 - DNA, 20 bp, Artificial sequence
  synthetic construct
ggagaagatccagcagcacc

SEQ ID NO: 37 - protein, 11 aa, Mus Musculus
HNQWNKPSKPK

SEQ ID NO: 38 - protein, 26 aa, Mus Musculus
QPHGGSWGQPHGGSWGQPHGGGWGQG

SEQ ID NO: 39 - protein, 46 aa, Mus Musculus
GNRYPPQGGTWGQPHGGGWGQPHGGSWGQPHGGSWGQPHGGGWGQG

SEQ ID NO: 40 - protein, 20 aa, Mus Musculus
MANLGYWLLALFVTMWTDVG

SEQ ID NO: 41 - protein, 36 aa, Mus Musculus
IHFGNDWEDRYYRENMYRYPNQVYYRPVDQYSNQNN

SEQ ID NO: 42 - protein, 4 aa, Hamster
MKHM

SEQ ID NO: 43 - protein, 26 aa, Mus Musculus
RSSSTVLFSSPPVILLISFLIFLIVG

SEQ ID NO: 44 - protein, 228 aa, Mus Musculus
MANLGYWLLALFVTMWTDVGLCKKRPKPGGWNTGGSRYPGQGSPGGNRYPPQGGTWGQPHGGGWGQPHGGSWGQPHGGSWGQPHGGGWGQGGGTHNQWNKPSKPKTNMKHMAGAAAAGAVVGGLGGYMLGSAMSRPMIHFGNDWEDRYYRENMYRYPNQVYYRPVDQYSNQNNFVHDCVNITIKQHTVTTTTKGENFTETDVKMMERVVEQMCVTQYQKESQAYYDGR